(19) United States
(12) Patent Application Publication
(10) Pub. No.: US 2008/0206095 A1
(43) Pub. Date: Aug. 28, 2008

(54) MICRO-ORGANISM REDUCTION IN LIQUID BY USE OF A METAL HALIDE ULTRAVIOLET LAMP

(76) Inventor: Robert E. Duthie, East Aurora, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/045,407

(22) Filed: Mar. 10, 2008

Related U.S. Application Data
(63) Continuation-in-part of application No. 11/190,089, filed on Jul. 26, 2005.
Application No. 11/190,089 is a continuation-in-part of application No. 09/903,825, filed on Jul. 11, 2001, now abandoned.

(51) Int. Cl. A61L 2/00

(52) U.S. Cl. 422/24

(57) ABSTRACT

A method and apparatus for disinfection/pasteurization of fluids. There is provided a mercury/gallium metal halide ultraviolet lamp enclosed within an ozone free metallic doped quartz envelope, an ozone free, metallic doped quartz enclosure for the lamp, an in-line stationary spiral or internal thread of single or multiple leads surrounding the enclosure, and a containment vessel having an inlet, an outlet and a chamber in fluid communication therewith defining a flow path for fluid to be disinfected/pasteurized. The lamp is operated at a wavelength range from about 100 nanometers to about 400 nanometers to introduce multiband ultraviolet radiation and minimal heat into the fluid with the enclosure preventing build up of ozone.

### CROSS REFERENCE TO RELATED APPLICATIONS

This application is a continuation-in-part of co-pending application Ser. No. 11/190,089 filed Jul. 26, 2005, and entitled “Micro-Organism Reduction In Liquid By Use Of A Metal Halide Ultraviolet Lamp”, which is a continuation-in-part of application Ser. No. 09/903,825 filed Jul. 11, 2001 and entitled “Micro-Organism Reduction In Liquid By Use Of A Metal Halide Ultraviolet Lamp”, the disclosures of which are hereby incorporated by reference.

### BACKGROUND OF THE INVENTION

This invention relates to the art of disinfection and pasteurization, and more particularly to a new and improved disinfection and pasteurization method and apparatus employing multiband ultraviolet light.

Since the detection of the micro-organism has increased within the food industry, non-thermal disinfection and pasteurization methods to reduce micro-organism contamination have also increased. Metal halide ultraviolet lamps have been employed in surface sterilization as described in U.S. Pat. No. 5,547,635 issued Aug. 20, 1996 and entitled “Sterilization Method and Apparatus,” the disclosure of which is hereby incorporated by reference.

### SUMMARY OF THE INVENTION

The present invention provides a method and apparatus employing non-thermal pasteurization utilizing technology involving surface sterilization with metal halide ultraviolet lamps. This unique non-thermal method of micro-organism reduction is achieved when a liquid is exposed to a high energy, metal halide, multiband ultraviolet lamp in an enclosed sealed chamber capable of allowing liquid flow into and out of a vessel. The radiation from the lamp will penetrate the liquid reducing the organism. The method comprises rapid heat transfer, titanium dioxide penetration, and multiband ultraviolet impregnation of the micro-organism within the liquid.

In addition, there is a lamp that emits UV-A (UV means ultraviolet light), UV-B and UV-C light at 2200-2250 A, 2660 A, 3710 A, 4440 A, and 4750 A. Riboflavin absorbs UV-A, UV-B and UV-C light at 2200-2250 A, 2660 A, 3710 A, 4440 A, and 4750 A. Absorption of ultraviolet at these wave lengths breaks apart the riboflavin radical, destroying certain elements and leaving “free” radicals which cannot replicate. Dynamic sterilization and disinfection operates at the aforementioned wave lengths and thereby destroys the cell by disassembling the Riboflavin radicals. The “free” radicals formed from the disassembling of riboflavin disrupt cellular metabolic activity and structure of the undesirable microorganisms, thereby killing the micro-organisms.

In addition, there is an apparatus comprising a vessel in which sterilization of the fluid is carried out has a structure that maximizes the destruction of the micro-organisms. The vessel comprises opposed ends, and inlet and an outlet. Internal to the vessel is at least one internal thread of single or multiple leads. The lead is spiral shaped. The lead surrounds a quartz tube that is disposed in an opening in the spiral thread. In one of the preferred embodiments, the lead abuts against the quartz tube and the vessel. There is an ultraviolet lamp disposed in the quartz tube for generating and emitting ultraviolet light, and this ultraviolet light passes through the quarts tubing. The lamp may be turned on and off. At least one turbulence bar is disposed in the vessel, and the turbulence bar causes turbulence in the flow of the fluid being sterilized which is flowing through the vessel. The turbulence bar causes the thorough mixing of the fluid being sterilized. The mixing ensures that the fluid all the fluid is exposed to the ultraviolet light emitted by the lamp. This results in the increased destruction of the micro-organisms, because the micro-organisms are not shielded by from the ultraviolet light.

The following detailed description of the invention when read in conjunction with the accompanying drawings, is in such full, clear, concise and exact terms as to enable any person skilled in the art to which it pertains, or with which it is most nearly connected, to make and use the same.

### BRIEF DESCRIPTION OF THE DRAWING

FIG. 1 is a side elevational view of the apparatus according to the present invention;

FIG. 2 is an end elevational view of the apparatus of FIG. 1;

FIG. 3 is a perspective view of the apparatus of FIG. 1 with the outer housing removed;

FIG. 3A is a front elevational view of a single lead.

FIG. 3B is a top plan view of the lead of FIG. 3A.

FIG. 3C is a is a sectional view of another embodiment of the lamp with a single lead.

FIG. 3D is a is a top plan view of the vessel of FIG. 3C showing the quarts tube, lamp and turbulence bars.

FIG. 3E is a is a front elevational view of the thread shown in FIG. 3C with turbulence bars.

FIG. 3F is a top plan view of the thread shown in FIG. 3E with turbulence bar.

FIG. 3G is a is a is a front elevation view of another embodiment of the lamp having a double lead.

FIG. 3H is a top plan view of the vessel of FIG. 3G showing the quarts tube, lamp and turbulence bars.

FIG. 3I is a front elevational view of the threads shown in FIG. 3G with turbulence bars.

FIG. 3J is a top plan view of the threads shown in FIG. 3I with turbulence bars.

FIG. 4 is a perspective view with parts removed of the apparatus of FIG. 1;

FIG. 5A is a fragmentary side elevational view of another form of the apparatus of the present invention;

FIG. 5B is a diagrammatic view further illustrating one of the units in the arrangement of FIG. 5B;

FIG. 6 is a graph providing definition of the ultraviolet light spectrum and the three wavelengths utilized in the present invention including germicidal ultraviolet light;

FIG. 7 is a spectral graph illustrating traditional germicidal ultraviolet light production;

FIG. 8 is a spectral graph illustrating multiband ultraviolet energy production utilized in the present invention;

FIG. 9 is a graph based on the data in Table 1;

FIG. 10 is a graph like that of FIG. 9 but giving additional information;

FIGS. 11A and 11B are diagrammatic views of a test set-up further illustrating the invention;

FIG. 12 is a graph illustrating results from comparisons using the set-up of FIGS. 11A and 11B;

FIGS. 13 and 14 are graphs presenting comparison data between a water purification system utilizing the invention and a water purification system utilizing conventional UV;

FIG. 15 is a schematic diagram illustrating particle path fluid dynamics in a flow processing apparatus;

FIG. 16 is a schematic diagram like FIG. 15 but for an alternative flow outlet arrangement;

FIG. 17 is a graph illustrating particle irradiation in the apparatus of FIG. 15;

FIG. 18 is a graph illustrating particle irradiation in the apparatus of FIG. 16;

FIG. 19 is a fluid particle path profile for the apparatus of the invention having the internal spiral geometry;

FIG. 20 is a graph illustrating fluid particle irradiation in the apparatus of the invention having the internal spiral geometry;

FIG. 21 is a diagrammatic view illustrating a pasteurizer incorporating the invention;

FIG. 22 is a schematic diagram illustrating particle path fluid dynamics in the pasteurizer of FIG. 21;

FIGS. 23A and 23B are graphs providing Reynolds Number plots associated with the illustration of FIG. 22;

FIG. 24 is a graph showing particle irradiation in the pasteurizer illustrated in FIGS. 21 and 22; and

FIGS. 25A and 25B are scanning electron microscope photographs further illustrating the invention.

### DETAILED DESCRIPTION OF THE INVENTION

The method and apparatus of the present invention employs non-thermal pasteurization utilizing research and technology involving surface sterilization with metal halide ultraviolet lamps. This unique non-thermal method of micro-organism reduction is achieved when a liquid is exposed to a high energy, metal halide, multiband ultraviolet lamp in an enclosed sealed chamber capable of allowing liquid flow into and out of a vessel. The radiation from the lamp will penetrate the liquid reducing the organism.

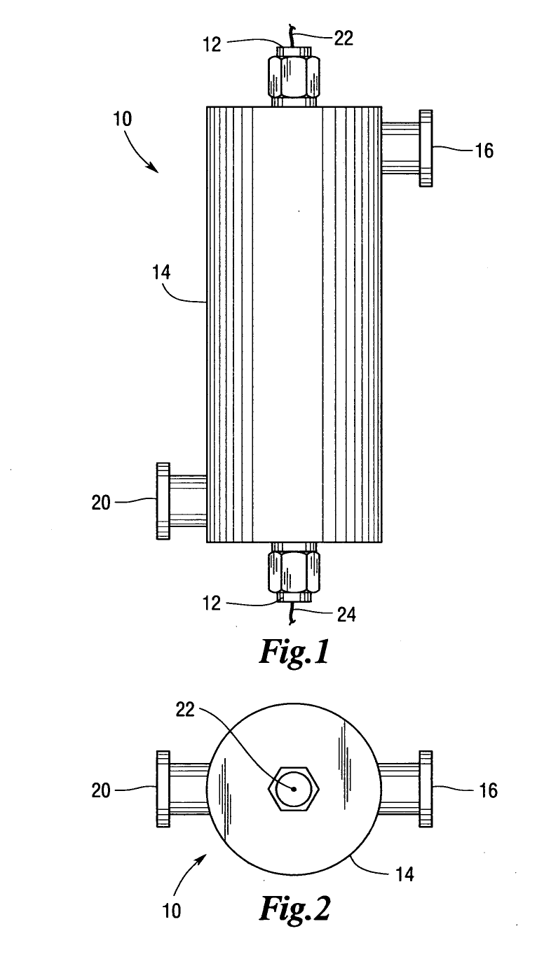

Referring to FIGS. 1-4, the apparatus for disinfecting/pasteurization of fluids 8 (also referred to herein as apparatus 8) comprises a lamp 10, with a metal halide configuration consisting of mercury and gallium, enclosed in an ozone free metallic doped quartz envelope, and having a wave length from about 100 to about 450 nanometers, is encapsulated in a second ozone free, metallic doped quartz tube 12. The metallic doping for the lamp envelope and tube 12 is titanium. Lamp 10 is similar to the lamp employed in the method and apparatus of the above-referenced U.S. Pat. No. 5,547,635. The quartz tube 12 is sealed in a vessel 14 comprising an inlet 16, cylindrical chamber 18 enveloping the tube and an in-line stationary, spiral, internal thread of single lead 23 or multiple leads 23a, 23b surrounding the tube, and an outlet 20.

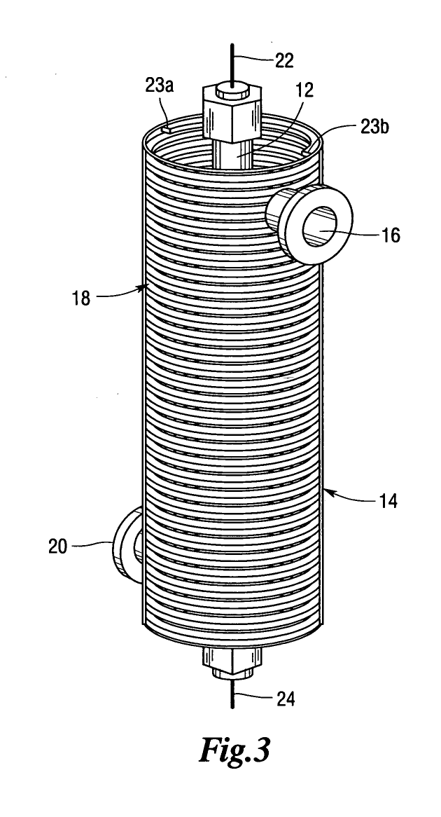

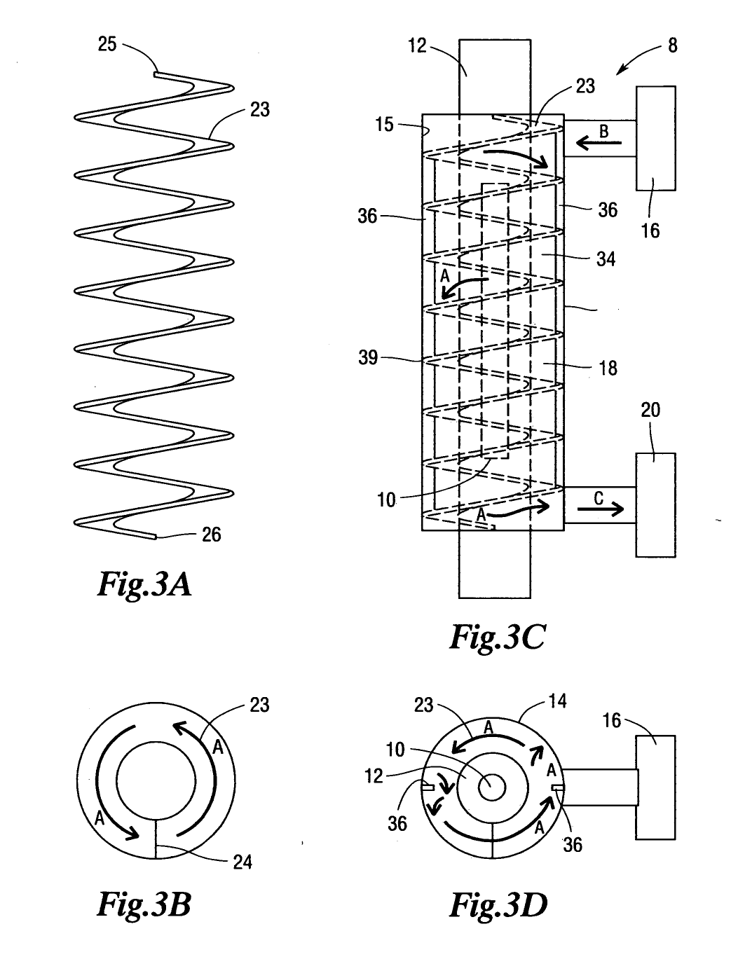

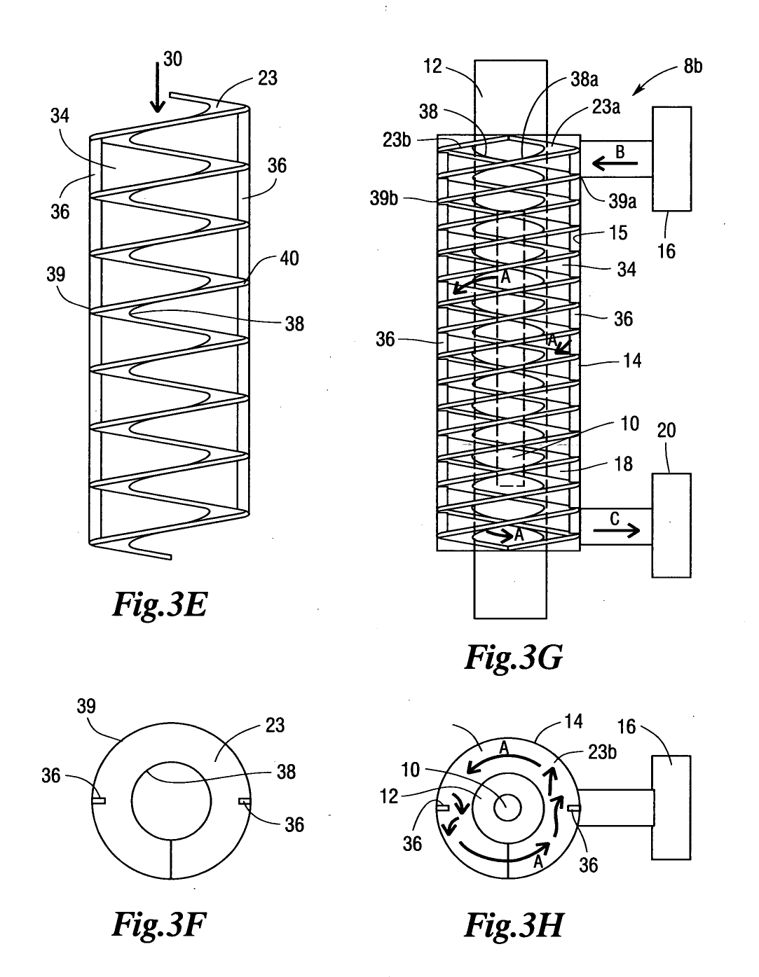

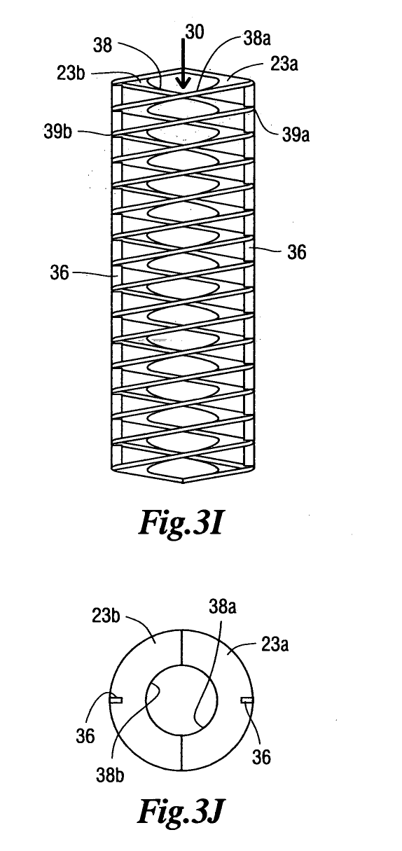

FIGS. 3A and 3B show a single lead 23 that is sized to be received in the vessel 14. The lead 23 is spiraled and has ends 25, 26. The lead 23 defines a tube opening 30 that is for receiving the metallic doped quartz tube 12. FIG. 3B shows flow arrows, designated A, which indicate the direction of fluid flow when the single lead 23 is assembly as part of the apparatus 8. Fluid is forced to flow along the spiral path of the lead 23 when the lead is positioned in the vessel 14 and the metallic doped quartz tube 12 is positioned in the tube receiving opening 30.

FIGS. 3C-3F show another preferred embodiment of the apparatus 8a. In this embodiment, apparatus 8a comprises a vessel 14 with an inlet 16 and an outlet 20, a single lead 23, and a lamp 10 positioned in the metallic doped quartz tube 12. The lead 23 is positioned in the chamber 18 of the vessel 14, and defines a tube opening 30 for receiving the quartz tube 12. The lead 23 has inner and outer edges 38, 39, respectively, and in one of the preferred embodiments the outer edge 39 abuts an inner surface 15 of the vessel 14, and the inner edge 38 abuts the metallic doped quartz tube 12. Fluid is thus unable to flow between the quartz tube 12 and the lead 23, and is unable to flow between the vessel 14 and the lead 23. A flow path 34 is defined between the lead 23, the inner surface 15 of the vessel 14 and the quartz tube 12, and the flow path 34 is spiraled due to the spiral shape of the lead 23.

Fluid enters through the inlet 16 as indicated by arrow B, and flows though the apparatus 8a. As the fluid flows through the vessel 14 through the inlet 16 and out the outlet 20 as indicated by arrow C, the fluid flows through the spiral flow path 34 in the direction of the arrows designated A. At least one turbulence bar 36 is joined to the lead 23, and FIGS. 3C-3F show two turbulence bars 36. In other embodiments there may be more than two turbulence bars 36. The turbulence bar 36 may be joined to outer edge 39 of the lead 23, as shown in FIG. 3E, and the turbulence bar 36 extends into the flow path 34. The outer edge 39 of the lead and the turbulence bar 36 may be flush, such that when the lead 23 is positioned in the vessel 14 the turbulence bar 36 and the lead 23 abut against the interior surface 15 of the vessel 14, as shown in FIG. 3C. The turbulence bar 36 advantageously disrupts the flow of the fluid as it moves through the flow path 32, and causes turbulent fluid flow. The turbulent flow of the fluid causes the fluid flow to become turbulent, as shown by the arrows designated A in FIG. 3D. This in turn forces the fluid to mix, such that fluid flows from a position proximal the interior surface 15 of the vessel 14 to a position proximal the quartz tube 12. This advantageously ensures that all of the fluid is exposed to the ultraviolet light emitted from the lamp 10, thereby destroying the micro-organisms in the fluid. An apparatus 8 with the turbulence bar 36 kills more micro-organisms as compared to a device not equipped with a turbulence bar 36, because in device without a turbulence bar the fluid proximal the vessel wall is not caused to flow in a direction towards the lamp 10, meaning it is possible that these micro-organisms may exit such a device in the same condition that the entered the device, i.e., healthy.

Shown in FIGS. 3G-3J is another preferred embodiment of the apparatus 8b. In this embodiment, apparatus 8b comprises a vessel 14 with an inlet 16 and an outlet 20, first and second leads 23a, 23b, respectively, and a lamp 10 positioned in a metallic doped quartz tube 12. The first and second leads 23a, 23b define a tube opening 30, and are positioned in the chamber 18 of the vessel 14. Each of the first and second leads 23a, 23b, has inner and outer edges 38, 38a, 39, 39a, respectively, and in one of the preferred embodiments the outer edges 39, 39a abut the inner surface 15 of the vessel 14, and the inner edges 38, 38a abut the metallic doped quartz tube 12. Fluid is thus unable to flow between the quartz tube 12 and the first and leads 23, 23a, and is unable to flow between the vessel 14 and the first and second leads 23, 23a. A flow path 34 is defined between the first and second leads 23, 23a the inner surface 15 of the vessel 14 and the quartz tube 12, and the flow path 34 is spiraled due to the spiral shape of the first and second leads 23, 23a.

Fluid enters through the inlet 16 as indicated by arrow B, and flows though the apparatus 8b. As the fluid flows through the vessel 14 through the inlet 16 and out the outlet 20 as indicated by arrow C, the fluid flows through the spiral flow path 34 in the direction of the arrows designated A. At least one turbulence bar 36 is joined to the lead 23, and FIGS. 3G-3J show two turbulence bars 36. In other embodiments there may be more than two turbulence bars 36. The turbulence bar 36 may be joined to outer edge 39 of the first and second leads 23a, 23b, as shown in FIG. 3I, and the turbulence bar 36 extends into the flow path 34. The outer edge 39 of the lead and the turbulence bar 36 may be flush, such that when the lead 23 is positioned in the vessel 14 the turbulence bar 36 and the lead 23 abut against the interior surface 15 of the vessel 14, as shown in FIG. 3C. The turbulence bar 36 advantageously disrupts the flow of the fluid as it moves through the flow path 32, and causes turbulent fluid flow. The turbulent flow of the fluid causes the fluid flow to become turbulent, as shown by the arrows designated A in FIG. 3H. This in turn forces the fluid to mix, such that fluid flows from a position proximal the interior surface 15 of the vessel 14 to a position proximal the quartz tube 12. One of the advantages of the turbulence bar 36 is that it mixes the fluid such that all of the fluid is exposed to the ultraviolet light emitted from the lamp 10, thereby destroying the micro-organisms in the fluid. An apparatus 8 with the turbulence bar 36 kills more micro-organisms as compared to a device not equipped with a turbulence bar 36, because in device without a turbulence bar the fluid proximal the vessel wall is not caused to flow in a direction towards the lamp 10, meaning it is possible that these micro-organisms may exit such a device in the same condition that the entered the device, i.e., healthy.

When the lamp 10 is ignited from an electronic ballast (not shown) in a circuit connected to the wires 22, 24 the lamp operates from about 100 nanometers to about 450 nanometers at a temperature ranging from about 400 degrees centigrade at the ends of the lamp to about 800 degrees centigrade at the center of the lamp. The afore-mentioned circuit can be similar to that shown in the above-referenced U.S. Pat. No. 5,547,635. The diameter of the vessel 14 is about twice the diameter of the tube 12 which in turn is twice the diameter of the lamp 10. The lamp 10 is approximately ½-inch diameter by about 16 inches in length for a ratio of about 100 watts per inch length at ½-inch diameter.

Liquid flow is dependent on the lamp output, spiral or threaded leads and pitch, and cylinder diameter. At constant velocity for a liquid, the internal spiral or thread increases the distance the liquid flows while in contact with the lamp and thereby increases the dwell time of the liquid in the lamp. Multiple threads increase mixing or turbulence of the liquid as it travels through the vessel. Flow rate for penetrations of a non-threaded vessel is about 0.1 gal/min./watt/vessel. This equates to a greater than five log reduction of micro-organism.

The process comprises rapid heat transfer and ultraviolet impregnation of the micro-organism within the liquid over the described period, and may also include titanium dioxide penetration. Multiband ultraviolet light and heat from lamp 10 are introduced to the liquid as the liquid follows along a flow path defined by the spiral through vessel 14. The quartz enclosure 12 allows ultraviolet light to be transmitted therethrough without appreciable buildup of ozone. The term rapid heat transfer used herein has the same general meaning as employed in describing the dry heat type of sterilization method. With rapid heat transfer, sterilization is time efficient with items drying quickly, in dry heat methods. In rapid heat transfer, as temperature increases, time decreases. It has been said that one way to get rapid heat transfer is to sterilize, not statically in a vessel but in a heat exchanger. The fluid is pumped continuously, and there is excellent energy economy by letting the hot sterilized medium exchange with the incoming medium. Applying the foregoing to the instant situation, the lamp 10, air and quartz tube 12 comprise the hot sterilized medium and the fluid or liquid flowing through vessel 14 comprises the exchange medium.

The unique non-thermal micro-organism reduction method additionally has implication in the medical industry.

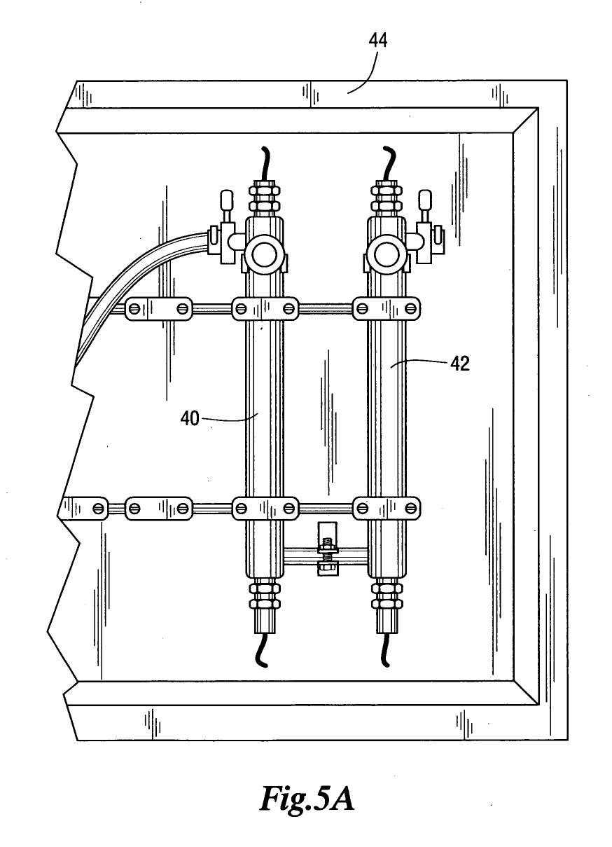

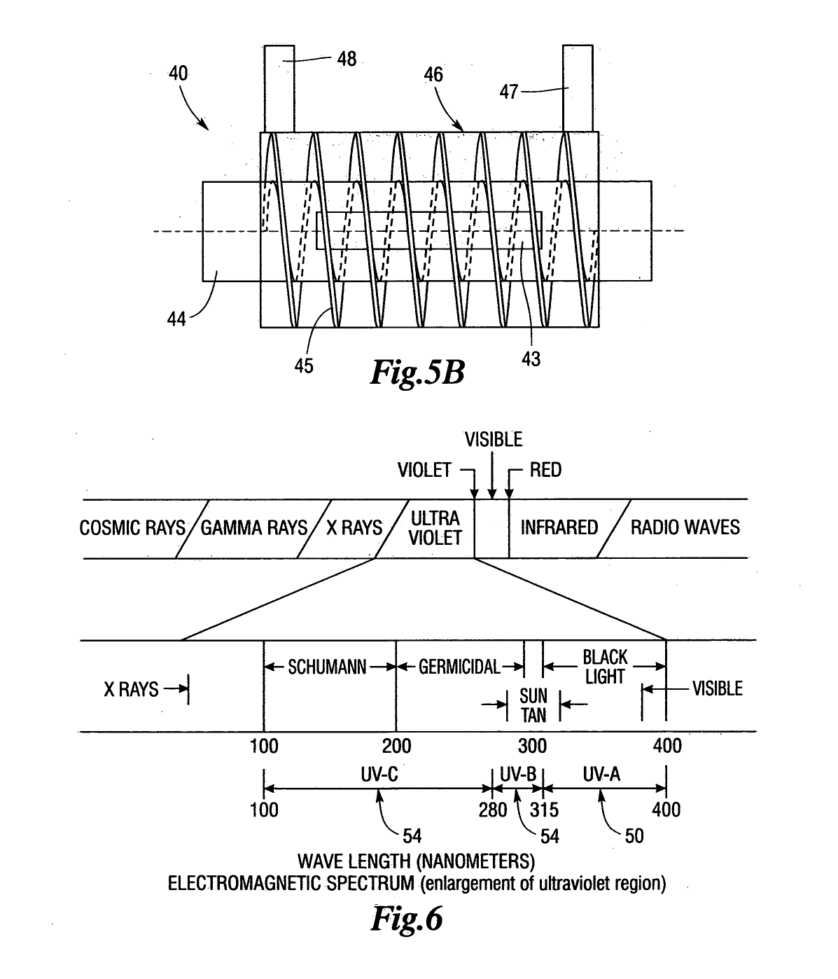

FIG. 5A illustrates another form of the apparatus of the invention wherein two units 40 and 42 are located within a housing 44 and connected in series. Each unit 40, 42 is similar to the apparatus shown in FIGS. 1-4 with an inlet on one end and an outlet on the other. FIG. 5B illustrates one of the units, for example unit 40, in more detail wherein a lamp 43, like lamp 10 of FIGS. 1-4, is within a quartz tube 44 surrounded by an auger 45 similar to threads 23 of FIGS. 1-4 which, in turn, is surrounded by vessel 46 having inlet 47 and outlet 48.

The method and apparatus of the present invention employs synergistic isogenous activated decontamination (a synergistic matrix of high energies simultaneously emitted from a single source) as a method of killing microorganisms by use of high intensity, broadband ultraviolet light combined with rapid heat transfer. This innovative and effective technology has particularly advantageous application to the food industry. However, the process has broad reaching potential applications far beyond the food industry, including the treatment of liquids, gasses, and solids.

The method and apparatus offer the following advantages over conventional methods of pasteurization or biological decontamination: processing speed, non-thermal, portable, nontoxic, no harmful radiation, electric and uncomplicated.

The method and apparatus of the present invention are further illustrated by the electromagnetic spectrum shown in FIG. 6. The specific wavelengths in the regions or bands identified UV-A, UV-B and UV-C which also are designated by the reference numerals 50, 52 and 54, show extremely high efficiency for producing micro-organism reduction or eradication when compared with the micro-organism reduction effects of current “state of the art” germicidal ultraviolet lamps operating only in the UV-C band designated 54 in FIG. 6. As shown in FIG. 6, band UV-A has a wavelength from about 315 nanometers to about 400 nanometers, band UV-B a wavelength from about 280 nanometers to about 315 nanometers and UV-C a wavelength from about 100 nanometers to about 280 nanometers.

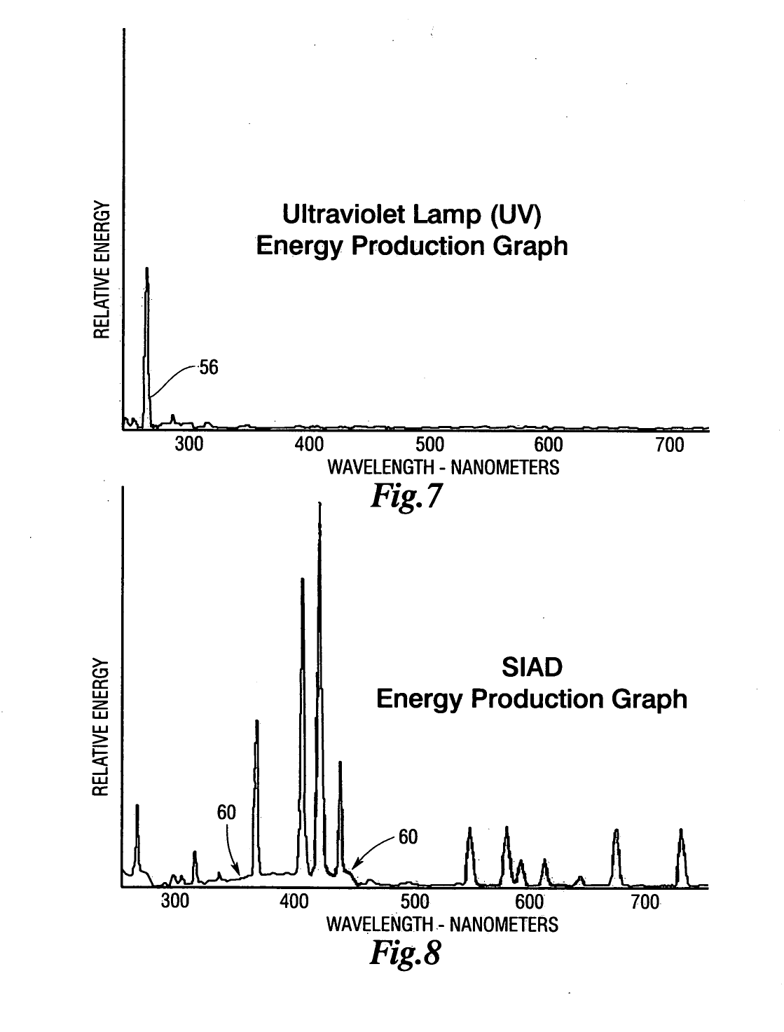

Current state of the art ultraviolet micro-organism reduction uses ultraviolet maps producing maximum energy only at 2,537 A or within the wavelength UV-C designated 54 in FIG. 6. In the spectral production graph of FIG. 7, the curve 56 illustrates the energy range of traditional, germicidal (UV-C) ultraviolet lamps such as these used in current water purification apparatus. The maximum bactericidal effectiveness of a germicidal (UV-C) ultraviolet lamp is manifested at 2,537 A, where 90% of exposed microorganisms are inactivated.

The mechanism of UV-C band germicidal action occurs as a result of the ultraviolet (UV-C) absorption at the 2,537 A wavelength by the nucleic acids or their components. This is the initial event in the chain of reactions leading to demise. Most of the damage elicited by UV-C light results in the formation of cyclobutane-type dimers between adjacent thymines in deoxyribonucleic acid. Similar dimers also form in lesser amounts between cytosines and between thymine-cytosine pairs. The dimers are extremely stable and they block the normal replication and transcription of the DNA. These irreversible changes compromise cellular function, which eventually leads to death. The amount of energy necessary to destroy microorganisms depends primarily on the sensitivity of the organism. Thus, ultraviolet (UV-C) light causes adjacent thymines (or cytosines) in DNA to dimerize.

Laboratory studies indicate that 90% inactivation of most viruses and bacteria is possible by current UV-C germicidal lamps. Surviving microorganisms are left in a weakened state, interfering with replication and increasing their susceptibility to other inactivation methods, including heat and chemical agents.

Traditionally used UV-C light at 2,537 A inactivates microorganisms by direct contact. Thus, microorganisms to be reduced would have to be directly exposed to the UV-C source. This could be termed “static sterilization and disinfection.” Sterilization is defined as the elimination or total destruction of microbial and viral life. Disinfection is the reduction of pathogenic microorganisms to a safe level by inhibiting cellular processes. Microorganisms may be shielded from direct UV-C by organic or inorganic matter. This protection from UV-C light is referred to as “screening and/or shadowing effect.” Screened microorganisms are not directly contacted by UV-C light. Therefore, screened microorganisms remain active following traditional UV-C irradiation.

Microorganism reduction using UV-C light is very limited and unreliable. However, the potential to sterilize does exist, as demonstrated by extensive research on airborne microbes. Additional reports support this claim, providing there is an unobstructed path of UV-C light to the target. For UV-C light to be considered a practical sterilization method, “shadow zones,” and “screening effects” must be eliminated.

Upon reviewing traditional germicidal UV-C ultraviolet microorganism reduction or “static sterilization and disinfection,” three important aspects of UV-C processing follow:
1. 90% activation of most microorganisms,
2. “screening and/or shadowing” affects the process, and
3. surviving microorganisms are left in a weakened state, interfering with replication and increasing their susceptibility to other inactivation methods, including heat and chemical agents.

The dilemma of traditional ultraviolet light (in the UV-C band) “static sterilization and disinfection” is overcome by means of a modified germicidal arc lamp providing simultaneous wavelength outputs of UV-A, UV-B and UV-C ultraviolet light previously described. In the spectral production graph of FIG. 8, curve 60 illustrates the energy range of the modified halide (UV-A, UV-B and UV-C) lamp used in the liquid purification apparatus of this invention. This improvement has generated the term “dynamic sterilization and disinfection.” Traditional germicidal, UV-C ultraviolet light sources lack the capacity to penetrate and cause molecular excitation by photon energy. The precise, simultaneous combination of UV-A, UV-B and UV-C ultraviolet light or “dynamic sterilization and disinfection” provides the capability of penetrating and causing molecular excitation. The excitation phenomenon involves the movement of organic and/or inorganic molecules, and the release of thermal energy.

Riboflavin (Vitamin B₂) occupies all cells including harmful microorganisms. Riboflavin will absorb UV-A, UV-B and UV-C light at 2200 A-2250 A, 2660 A, 3710 A, 4440 A, and 4750 A. Absorption of ultraviolet at these wave lengths breaks apart the Riboflavin radical, destroying certain elements and leaving “free” radicals which cannot replicate. Dynamic sterilization and disinfection operates at the aforementioned wave lengths and thereby destroys the cell by disassembling the Riboflavin radicals. The “free” radicals formed from the disassembling of Riboflavin disrupt cellular metabolic activity and structure. In particular, this “free” radical operation interferes with the quality control function of protein in the cell, thereby rendering the cell susceptible to either self destruction or destruction by an external force or effect.

Riboflavin acts in living organisms as a coenzyme, flavin adenine dinucleotide (FAD). FAD is part of the mitochondrial electron transport chain and is the coenzyme for glutathione reductase (GR), an enzyme involved in the regeneration of glutathione. Glutathione is critical in reactivating Vitamin C. When Vitamin E is inactivated by neutralizing free radicals as those formed by the multiband ultraviolet light (UV-A, B, & C) disassembling Riboflavin, Vitamin C regenerates Vitamin E back to full activity. Vitamin E prevents oxidation of unsaturated fatty acids by trapping “free radicals.” This stabilizes and protects cell membranes and tissues sensitive to oxidation. Vitamin E has synergistic effects with Vitamin C, gluthathione, and other antioxidants. Thus, the disassembling of Riboflavin by multiband ultraviolet light (UV-A, B, & C) inactivates the rebuilding mechanism for the cell membrane, causing the cell membrane to be vulnerable to destruction from various energy sources such as heat and light. With the destruction of the cell membranes, the DNA chain, unprotected within the cellular structure, is exposed and vulnerable.

Utilizing a metal halide, multiband ultraviolet lamp with output ratios of about 35% UV-A, 40% UV-B and 25% UV-C according to the invention, the dynamic sterilization, and disinfection technology dramatically outperformed the traditional germicidal ultraviolet (UV-C) technology in microorganism reduction. The foregoing percentages apply to the distribution of the total relative energy (microwatts/cm²/sec or Joules/m²/sec) output from the lamp through the entire UV spectral range (UV-A, UV-B and UV-C). In particular, the relative energy distribution of the lamp according to the invention is approximately 35% MV/cm²/sec in UV-A (315-400 nm), 40% MW/cm²/sec in UV-B (280-315 nm) and 25% MW/cm²/sec in UV-C (100-280 nm). The dynamic sterilization and disinfection lamps according to the invention operate in the broadband UV spectrum (1000 A to 4000+A). These multiband lamps output high energy throughout the UV spectrum range. In addition, during operation the temperature at the center of the multiband lamp is in excess of 500 C. Utilizing the multiband lamp, this unique synergism of traditional UV, high energy, broad band UV and heat transfer operate simultaneous on microorganisms. The total processing mechanism for dynamic sterilization and disinfection according to the present invention is termed synergistic isogenous activated decontamination (SIAD).

By way of example, a mercury/gallium lamp found to perform satisfactorily in the invention is commercially available from Voltarc Technologies Inc. of Fairfield, Conn. under part number 18522 UY15C/8FR/3654. The lamp glass is titanium doped silica quartz, and the lamp contains 20 Torr Argon gas, gallium in the amount of 1 Mg. and Mercury in the amount of 72 Mg. Data for an illustrative ⅜ inch diameter by 8-inch length version of the lamp is presented in Table 1, it being understood that other diameter and length versions of the lamp can be employed. In Table 1, the data is organized with the far left column being the starting wavelength in nanometers for the row, and each number in the row is the energy (microwatts/sq.cm.×0.02 at 1 meter) for incremental wavelengths. For example, in the first row 2030 is the energy at 250 nanometers, 3807 the energy at 251 nanometers and proceeding to the right-hand end of the row, 759 is the energy at 259 nanometers. The far right column in Table 1 is the total energy for the preceding 10 wavelengths. Thus, 12410 is the sum of all ten entries in the first row. In Table 1, x represents energy and y represents wavelength.

TABLE 1

| | |
|---|---|
| MERCURY/GALLIUM METAL HALIDE LAMP | 1500 WATT |
| ⅜ inch Diameter × 8 inch Length | 500 ARC VOLT |
| Titanium Doped, Ozone-Free Quartz | X = 0.2561, Y = 0.2418 |
| MULTIPLY BY 0.02000000 FOR MICROWATTS/SQ.CM./NM., AT 1.000 METERS | |

| AVL | | | | | | | | | | | SUM |
|---|---|---|---|---|---|---|---|---|---|---|---|
| | | | | | | | | | | | /10 NM |
| 250 | 2030 | 3807 | 554 | 666 | 725 | 1265 | 780 | 1113 | 711 | 759 | 12410 |
| 260 | 754 | 369 | 236 | 493 | 734 | 1543 | 1241 | 604 | 311 | 332 | 6617 |
| 270 | 420 | 636 | 662 | 465 | 374 | 367 | 311 | 246 | 164 | 343 | 3990 |
| 280 | 512 | 480 | 323 | 194 | 174 | 270 | 652 | 1648 | 2248 | 1715 | 8215 |
| 290 | 989 | 603 | 581 | 1143 | 2590 | 3382 | 3229 | 2429 | 1363 | 823 | 17132 |
| 300 | 714 | 1194 | 1767 | 1316 | 527 | 327 | 273 | 228 | 226 | 214 | 6785 |
| 310 | 269 | 670 | 2402 | 3572 | 2094 | 589 | 284 | 221 | 194 | 184 | 10479 |
| 320 | 169 | 158 | 151 | 147 | 158 | 166 | 155 | 200 | 235 | 189 | 1728 |
| 330 | 143 | 144 | 164 | 372 | 654 | 476 | 186 | 171 | 198 | 190 | 2698 |
| 340 | 136 | 137 | 131 | 144 | 144 | 141 | 133 | 132 | 134 | 136 | 1368 |
| 350 | 133 | 131 | 129 | 132 | 134 | 134 | 139 | 155 | 168 | 161 | 1416 |
| 360 | 150 | 166 | 203 | 416 | 2817 | 6554 | 5492 | 2008 | 555 | 288 | 18647 |
| 370 | 241 | 224 | 231 | 230 | 235 | 220 | 195 | 178 | 166 | 181 | 2101 |
| 380 | 181 | 192 | 212 | 209 | 186 | 190 | 199 | 193 | 183 | 191 | 1936 |
| 390 | 218 | 224 | 194 | 187 | 183 | 181 | 191 | 203 | 207 | 226 | 2014 |
| 400 | 271 | 437 | 1926 | 7096 | 10849 | 8302 | 4311 | 2350 | 1415 | 713 | 37670 |
| 410 | 426 | 352 | 330 | 340 | 418 | 676 | 2985 | 9889 | 12554 | 8438 | 36410 |
| 420 | 5373 | 3270 | 1705 | 954 | 658 | 524 | 446 | 400 | 365 | 342 | 14038 |
| 430 | 335 | 336 | 325 | 400 | 766 | 3843 | 6504 | 3260 | 887 | 521 | 17177 |
| 440 | 437 | 417 | 413 | 414 | 388 | 324 | 253 | 196 | 161 | 139 | 3141 |
| | | | | | | | | | | | /20 NM |
| 450 | 125 | 106 | 98 | 97 | 101 | 109 | 112 | 105 | 91 | 81 | 2050 |
| 470 | 80 | 80 | 78 | 77 | 76 | 76 | 80 | 81 | 83 | 90 | 1602 |
| 490 | 106 | 115 | 111 | 110 | 115 | 117 | 110 | 85 | 65 | 56 | 1980 |
| 510 | 52 | 52 | 51 | 50 | 48 | 48 | 48 | 48 | 50 | 49 | 993 |
| 530 | 51 | 57 | 71 | 78 | 76 | 92 | 362 | 2232 | 3660 | 2048 | 17452 |
| 550 | 357 | 136 | 88 | 72 | 63 | 59 | 57 | 54 | 53 | 55 | 1988 |
| 570 | 66 | 116 | 599 | 1690 | 2131 | 1225 | 331 | 102 | 100 | 158 | 13036 |
| 590 | 144 | 79 | 53 | 50 | 49 | 48 | 47 | 48 | 48 | 53 | 1237 |
| 610 | 56 | 52 | 48 | 47 | 47 | 48 | 51 | 51 | 50 | 48 | 998 |
| 630 | 48 | 49 | 49 | 61 | 123 | 166 | 116 | 61 | 50 | 49 | 1544 |
| 650 | 48 | 48 | 49 | 48 | 49 | 49 | 48 | 51 | 54 | 177 | 1241 |
| 670 | 375 | 279 | 78 | 53 | 50 | 51 | 52 | 50 | 51 | 60 | 2202 |
| 690 | 81 | 74 | 56 | 52 | 52 | 50 | 53 | 53 | 56 | 61 | 1173 |
| 710 | 59 | 52 | 54 | 54 | 51 | 53 | 53 | 53 | 53 | 53 | 1070 |
| 730 | 53 | 51 | 51 | 53 | 54 | 54 | 54 | 54 | 56 | 55 | 1069 |
| 750 | 53 | 52 | 52 | 51 | 51 | 51 | 53 | 58 | 67 | 63 | 1104 |
| 770 | 66 | 69 | 65 | 57 | 59 | 62 | 57 | 55 | 57 | 45 | 1184 |
| 790 | 53 | 50 | 53 | 50 | 46 | 48 | | | | | 599 |

GRAND TOTAL = 2584.9
NIOSH Wt'd Spectral Irrad. (E, Eff., 250-315 nm) = 307.25
MPET (Max. permissible exposure time) = 9,7641 seconds

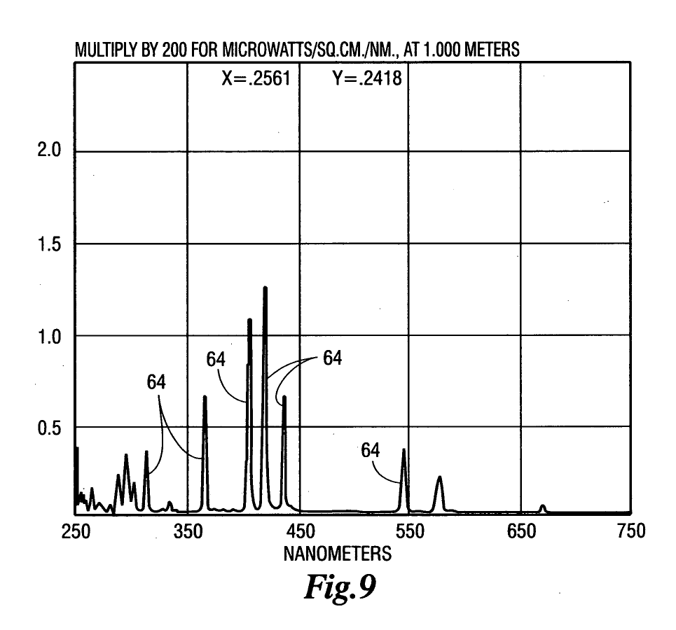

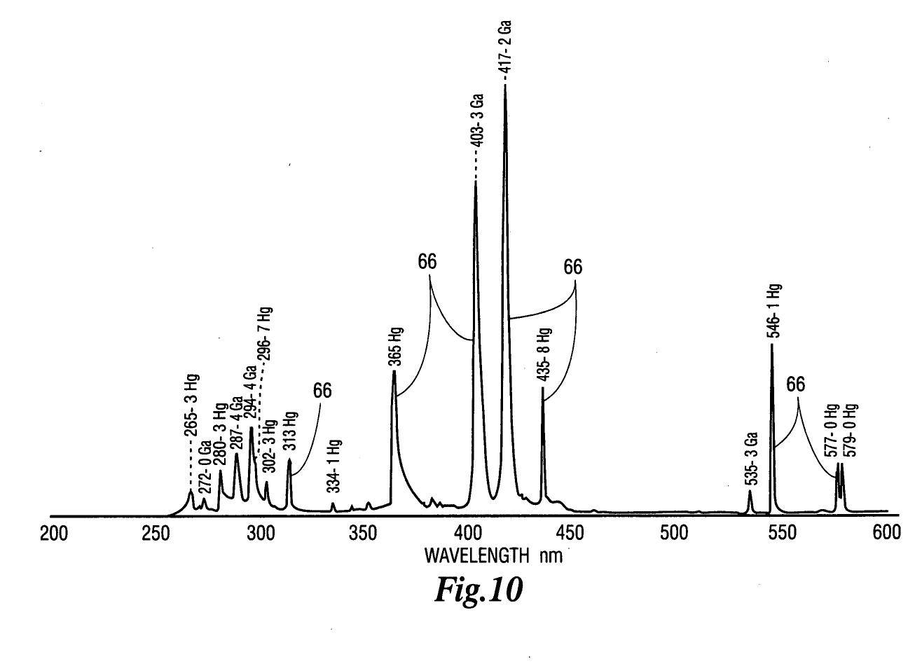

The graph of FIG. 9 containing plot 64 is prepared from the data of Table 1. The graph of FIG. 10 containing plot 66 is like that of FIG. 10 but indicates the chemicals in the lamp which give the spikes at the specific wavelengths.

Thus, to summarize, if the ultraviolet wavelengths (UV-A, UV-B, & UV-C) are combined in the proper proportions (approximately 35% UV-A, 40% UV-B, and 25% UV-C) and administered simultaneously to the micro-organism, in accordance with the method of the invention, the organism is reduced faster, more completely and without the complications (i.e. shadowing) attributed to traditional ultraviolet UV-C processing. Simply presented, when UV-A, UV-B and UV-C are (1) simultaneously and (2) in the correct proportionality administered to a micro-organism, the organism's cellular membranes are destroyed by the formation of “free radicals”, leaving the cell's DNA strand exposed and extremely vulnerable to UV-C. By this process, the (1) protective and (2) regenerative mechanisms of the cells of the micro-organism are destroyed simultaneously causing the cells and any cellular functions to immediately cease. The presence of rapid heat transfer, introduced to this process by infrared wavelengths (above 4000 A) from the UV lamp, increases the described, cellular destruction process.

The apparatus of the invention plays a critical role in the foregoing operation. First, the lamp 10 must be able to administer the stated proportionality of UV-A, UV-B and UV-C simultaneously. Second, the vessel 14 which the liquid passes through must maintain the liquid's contact with the lamp's ultraviolet rays for a time dependent on the lamp's intensity and the fluid's velocity through the vessel. The mercury gallium metal halide lamp 10 delivers the required proportionalities of UV-A, UV-B, and UV-C ultraviolet plus infrared light. The vessel 14 has an internal thread 23 extending the length of the vessel and protruding from the inner wall. This internal thread increases the distance the fluid must travel between the inlet and the outlet of the vessel. Maintaining a constant fluid velocity, the increased distance dictated by the vessel's internal thread causes an increase in the dwell time between the liquid and lamp. Furthermore, by increasing the internal thread's leads (i.e. double thread, triple thread etc.) and maintaining the same pitch, the required dwell time between liquid and lamp can be reduced due to the turbulence of the liquid caused by the multiple threads.

Successful animal and human clinical studies involving implanted devices have demonstrated no adverse reactions following synergistic inactivation and disinfection processing and complete compatibility with living cells. Dynamic sterilization and disinfection or synergistic isogenous activated decontamination (SIAD) has been examined with respect to the food processing industry. The process of the invention was found complimentary to both liquids and solids in microorganism reduction without altering product chemistry or physical composition.

The invention is illustrated further by the following comparison of the bactericidal effect of a photonic lamp 10 of the invention with a conventional ultraviolet lamp on a strain of Escherichia coli. The term “photonic lamp” refers to the multi-band or multi-spectrum UV lamp of the invention, as differentiated from conventional germicidal UV lamps operating only in the UV-C range. In particular a comparison was made of the bactericidal effect of a photonic lamp 10 of the invention with an ultraviolet (UV) light source at various distances from bacterial cell suspensions of Escherichia coli (E. coli) at varying sample depths.

The conventional ultraviolet lamp was one commercially available from Water Purification, Inc. under the designation Ultraviolet Lamp #FB01 and having the following specifications: range: UVC; watts: 22; voltage: 117 AC; amps: 0.19; and physical dimensions: 20 inch lighted length×0.625 inch diameter.

The lamp of the invention was an 8″ photonic lamp having the following specifications: range: UVA, UVB, and UVC; watts: 1500; voltage: 117 AC; amps: 13; and physical dimensions: 8 inch lighted length×0.375 inch diameter.

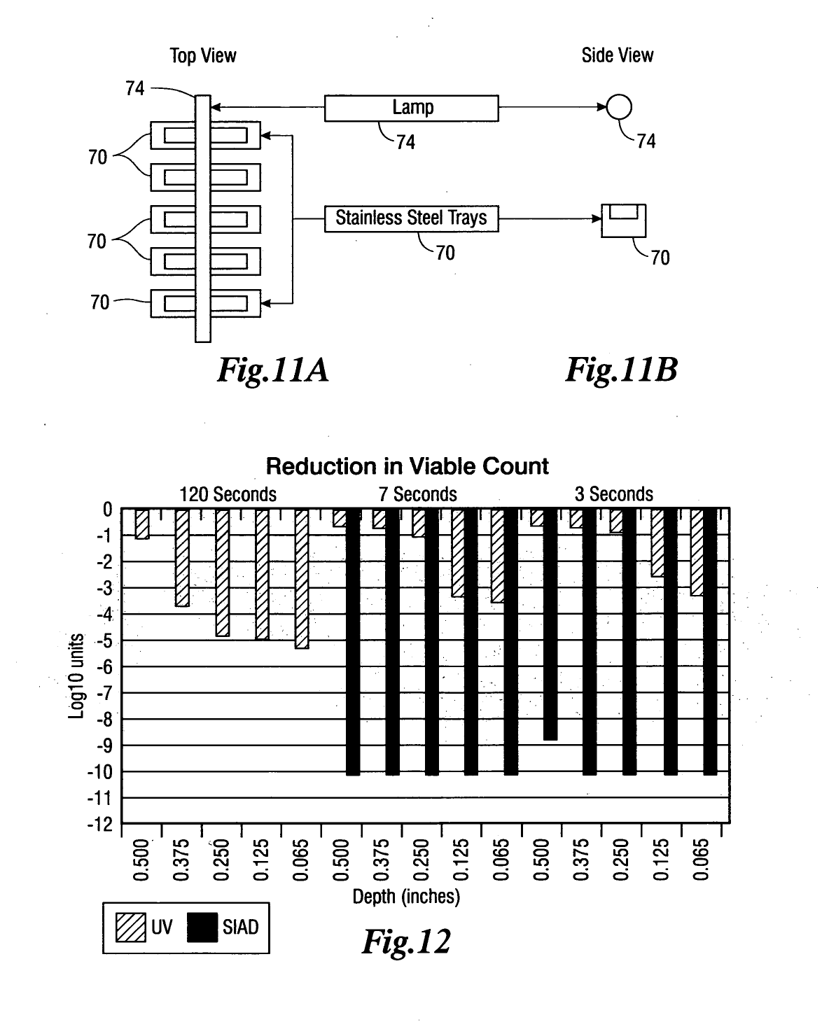

Escherichia coli is an enteric, gram-negative, motile, non-spore forming rod commonly used as an indicator organism for fecal contamination of water supplies. In this study a strain of E. coli (ATCC #47056) was grown on the surface of 10 Tryptic Soy Agar (TSA) plates and harvested after 18 hours of incubation at 37° C. in 5.0% CO₂ in air. The cells were suspended in sterile full-strength “Ringers” solution and placed in stainless steel trays 70 shown in FIG. 11A of varying capacities and depths as indicated by the entries in Tables 2 and 3. Saline was used rather than water to diminish the effect of cell lyses due to osmotic pressure. The trays were then placed directly below each lamp as shown in FIGS. 11A and 11B and exposed for periods of 3 and 7 seconds at a distance (R) of 2.25 inches from the lamp. In FIGS. 11A and 11B the reference numeral 74 represents both the conventional lamp and the lamp of the invention, the former being positioned in the arrangement of FIGS. 11A and 11B for the trials documented in Table 3 and the latter being positioned in that arrangement for the trials documented in Table 2. A third trial was conducted with the UV light using an exposure of 120 seconds. All trials included five varying depths (B) of cell suspension as indicated in Tables 2 and 3. Voltage, lamp temperature, and fluid temperatures at both the start and end of exposure were recorded at each trial for the photonic lamp of the invention throughout the study.

Following exposure, an aliquot of cell suspension was removed from each tray, plated onto TSA and incubated at 37° C., in air, for 24 hours. The recoverable cell counts were determined by plating appropriate dilutions onto TSA and compared to those of unexposed cell suspensions of E. coli. The results are summarized in Tables 2 and 3.

TABLE 2
Effect of Photonic Lamp of the Invention on 18 hr Culture of E. coli

| | | | | | | | | | Viable |
|---|---|---|---|---|---|---|---|---|---|
| | | Exposure | R¹ | B² | | Lamp | Fluid Temp | | Count |
| Trial# | Sample# | (sec.) | (in.) | (in.) | Voltage | Temp. | Start | End | CFU/ml |
| | 0 | Unexposed | N/A | N/A | N/A | N/A | N/A | N/A | 9.47E+09 |
| 1 | 1 | 7 | 2.25 | 0.500 | 523 | 150° C. | 21° C. | 22° C. | 0.00E+00 |
| | 2 | 7 | 2.25 | 0.375 | 523 | 150° C. | 21° C. | 22° C. | 0.00E+00 |
| | 3 | 7 | 2.25 | 0.250 | 523 | 150° C. | 21° C. | 22° C. | 0.00E+00 |
| | 4 | 7 | 2.25 | 0.125 | 523 | 150° C. | 21° C. | 22° C. | 0.00E+00 |
| | 5 | 7 | 2.25 | 0.065 | 523 | 150° C. | 21° C. | 22° C. | 0.00E+00 |
| 2 | 6 | 3 | 2.25 | 0.500 | 525 | 160° C. | 21° C. | 21° C. | 2.03E+01 |
| | 7 | 3 | 2.25 | 0.375 | 525 | 160° C. | 21° C. | 21° C. | 0.00E+00 |
| | 8 | 3 | 2.25 | 0.250 | 525 | 160° C. | 21° C. | 21° C. | 0.00E+00 |
| | 9 | 3 | 2.25 | 0.125 | 525 | 160° C. | 21° C. | 21° C. | 0.00E+00 |
| | 10 | 3 | 2.25 | 0.065 | 525 | 160° C. | 21° C. | 21° C. | 0.00E+00 |

TABLE 3
Effect of Ultraviolet Lamp #FB01 on 18 hr Culture of E. coli

| | | | | | | | | | Viable |
|---|---|---|---|---|---|---|---|---|---|
| | | Exposure | R¹ | B² | | Lamp | Fluid Temp | | Count |
| Trial# | Sample# | (sec.) | (in.) | (in.) | Voltage | Temp. | Start | End | CFU/ml |
| | 0 | Unexposed | N/A | N/A | N/A | N/A | N/A | N/A | 1.02E+10 |
| 1 | 1 | 120 | 2.25 | 0.500 | 117 | 25.7 | 25.0 | 25.0 | 1.06E+09 |
| | 2 | 120 | 2.25 | 0.375 | 117 | 25.7 | 25.0 | 25.0 | 2.85E+06 |
| | 3 | 120 | 2.25 | 0.250 | 117 | 25.7 | 25.0 | 25.0 | 1.77E+05 |
| | 4 | 120 | 2.25 | 0.125 | 117 | 25.7 | 25.0 | 25.0 | 1.48E+05 |
| | 5 | 120 | 2.25 | 0.065 | 117 | 25.7 | 25.0 | 25.0 | 7.32E+04 |
| 2 | 6 | 7 | 2.25 | 0.500 | 117 | 25.7 | 24.3 | 24.3 | 3.25E+09 |
| | 7 | 7 | 2.25 | 0.375 | 117 | 25.7 | 24.3 | 24.3 | 2.85E+09 |
| | 8 | 7 | 2.25 | 0.250 | 117 | 25.7 | 24.3 | 24.3 | 1.26E+09 |
| | 9 | 7 | 2.25 | 0.125 | 117 | 25.7 | 24.3 | 24.3 | 6.71E+06 |
| | 10 | 7 | 2.25 | 0.065 | 117 | 25.7 | 24.3 | 24.3 | 4.07E+06 |
| 3 | 11 | 3 | 2.25 | 0.500 | 117 | 25.7 | 24.5 | 24.5 | 3.41E+09 |
| | 12 | 3 | 2.25 | 0.375 | 117 | 25.7 | 24.5 | 24.5 | 2.68E+09 |
| | 13 | 3 | 2.25 | 0.250 | 117 | 25.7 | 24.5 | 24.5 | 1.79E+09 |
| | 14 | 3 | 2.25 | 0.125 | 117 | 25.7 | 24.5 | 24.5 | 3.90E+07 |
| | 15 | 3 | 2.25 | 0.065 | 117 | 25.7 | 24.5 | 24.5 | 7.11E+06 |

¹“R” = distance from the center of the lamp to the surface of the bacterial cell suspension.
²“B” = depth of sample.

Table 2 shows that the voltage and temperature of the photonic lamp of the invention remained relatively stable throughout the study. The voltage for trials 1 and 2 were 523 and 525 volts respectively while the lamp temperature ranged from 150° C. to 160° C. The fluid temperature remained stable and did not exceed 22° C., well within a range that would not alter viable cell counts.

Total viable count was reduced to undetectable levels in nine of ten samples exposed to the photonic lamp of the invention with an average cell recovery rate of 2.03 colony forming units (cfu)/ml (Table 2). This was in sharp contrast to the UV lamp exposures that resulted in an average cell recovery of 1.09×10⁹ cfu/ml (Table 3) representing an average drop in viable count of 2.35 log units compared to an average drop of almost 10 log units for the photonic lamp of the invention as further illustrated in the chart of FIG. 12. A decrease in bacterial kill was observed for the UV light samples with increased sample depth for all three time exposure times, while only one sample exposed to the photonic lamp of the invention.

The photonic lamp of the invention provided greater sample penetration and bactericidal effect than the ultraviolet light tested. This data suggests that the photonic lamp of the invention may be useful in the control and eradication of viable E. coli from water and other fluids.

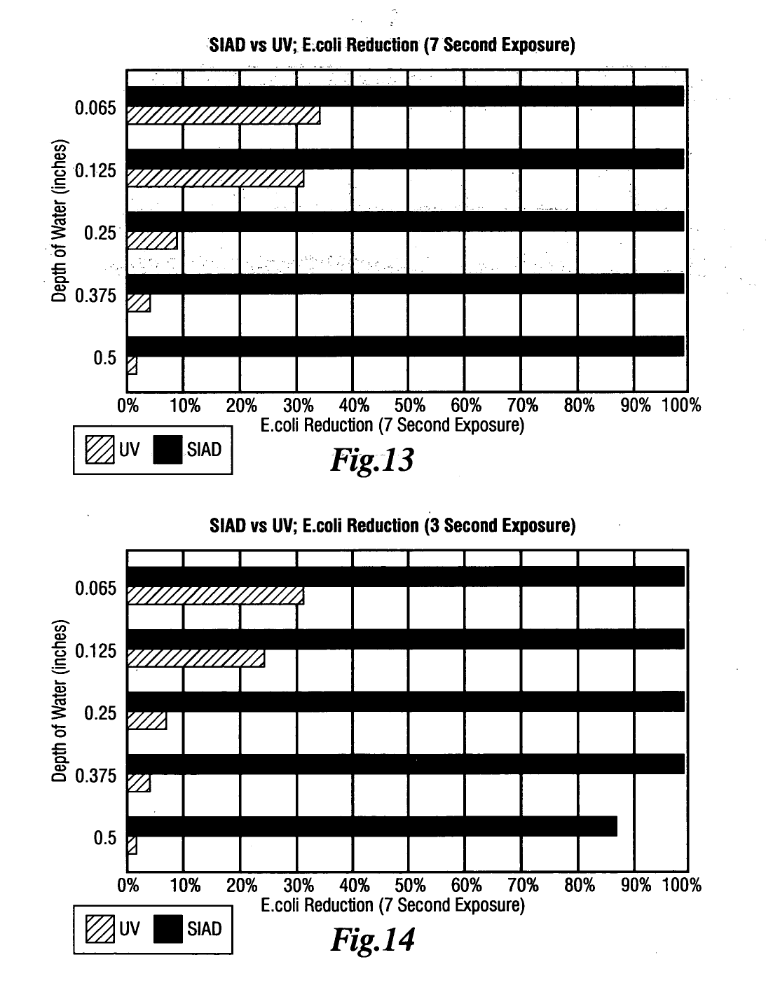

FIGS. 13 and 14 are charts comparing a water purification system utilizing the method and apparatus of the invention with a standard household UV-C water purification. In particular, FIG. 13 compares the two during a 7-second exposure, showing the extent of E. coli reduction and FIG. 14 compares the two during a 3 second exposure, showing the extent of E. coli reduction. By way of example, an illustrative water purification system utilizing the method and apparatus of the invention can be implemented using the arrangement of FIGS. 5A and 5B.

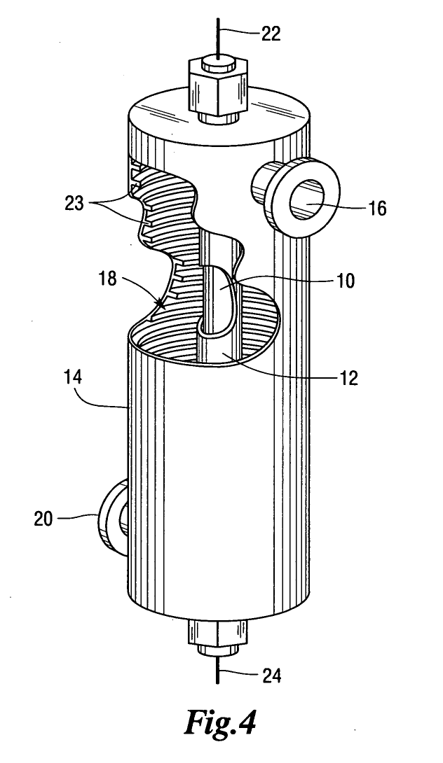

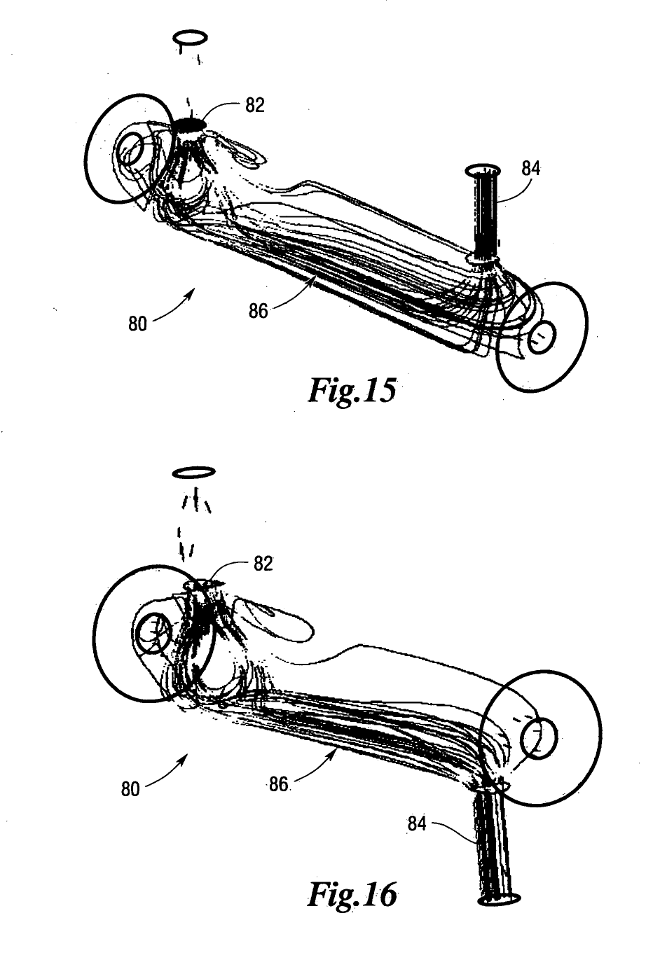

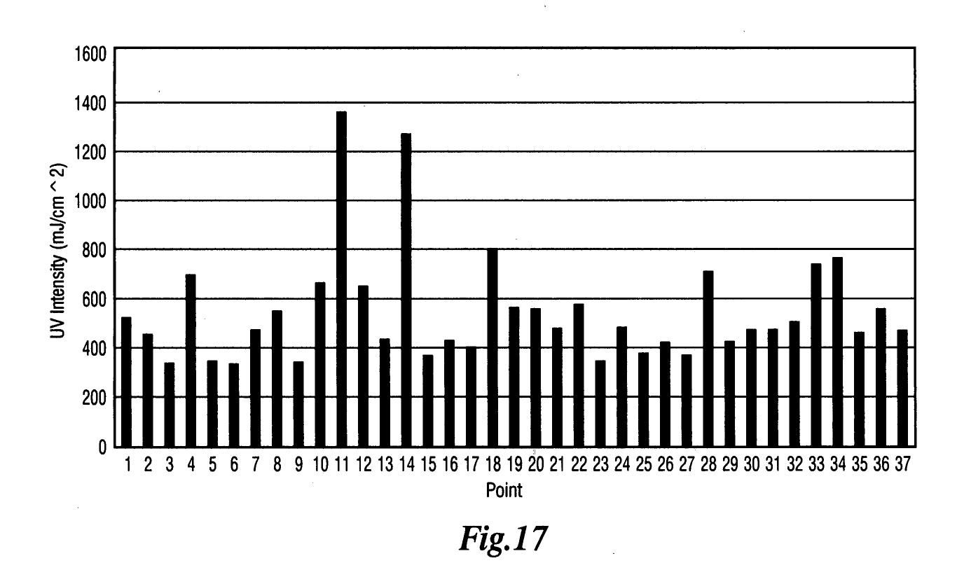

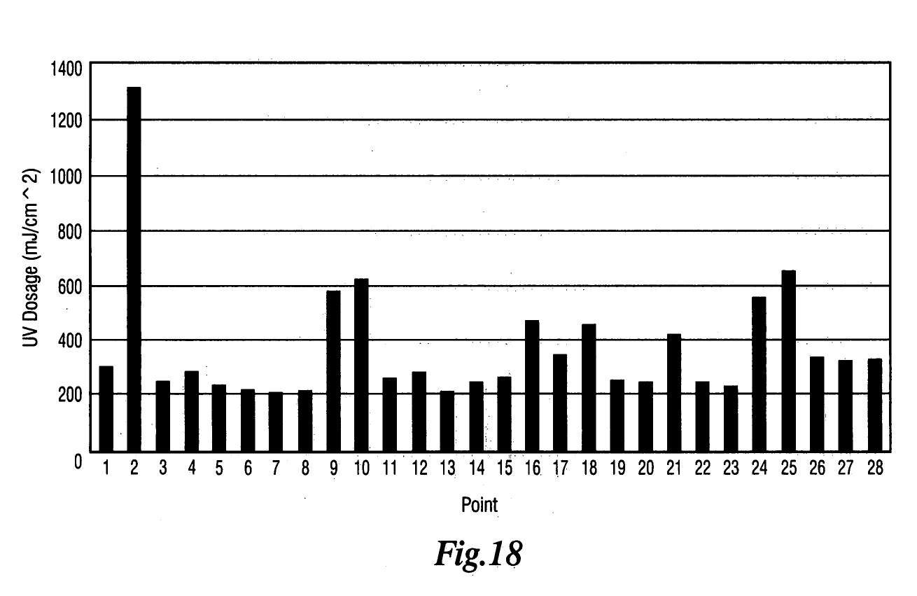

The method and apparatus of the invention illustrated in FIGS. 1-5 may be viewed as incorporating a flow processing mode of operation, in contrast to a relatively static, batch processing mode of operation. In addition, with the internal spiral geometry provided by threads 23 shown in FIGS. 3 and 4 and auger 45 shown in FIG. 5B, fluid particle irradiation advantageously is controlled and equalized rather than being random. In particular, FIG. 15 is a schematic diagram illustrating particle path fluid dynamics in a flow processing apparatus 80 similar to the apparatus shown in FIGS. 1-5 but without the internal spiral geometry, i.e. without threads 23 or auger 45. The apparatus 80 has an inlet 82 and outlet 84, and lines 86 represent the fluid particle flow paths from inlet 82 through the apparatus to the outlet. FIG. 16 is a schematic diagram like FIG. 15 wherein the components are identical and the outlet 84 is oriented in a direction opposite that shown in FIG. 15. FIGS. 17 and 18 are graphs illustrating particle irradiation in the arrangements of FIGS. 15 and 16, respectively, and it can be seen from these graphs that the fluid particle irradiation is random.

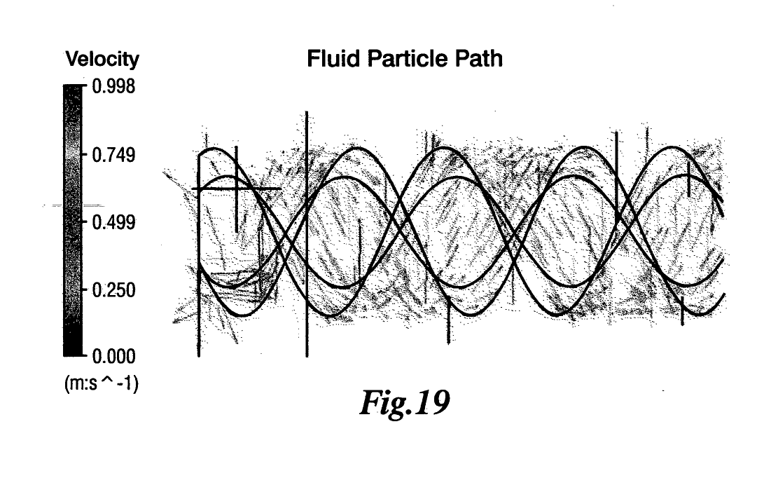

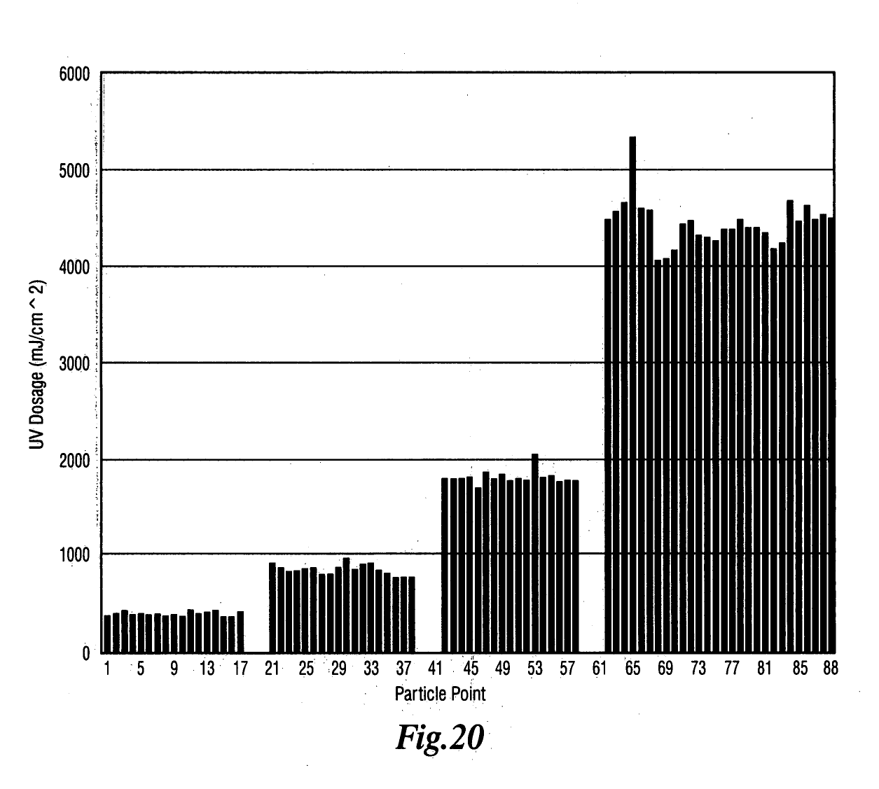

In arrangements incorporating the internal spiral geometry, illustrated for example in FIGS. 3, 4 and 5B, particle irradiation is controlled and equalized as compared to random irradiation associated with non-spiral arrangements. FIG. 19 is a fluid particle path velocity profile for the apparatus of the invention incorporating the internal spiral geometry, i.e. the threads 23 in the apparatus of FIGS. 1-4 and the auger 45 in the apparatus shown in FIG. 5B. FIG. 20 is a graph illustrating fluid particle irradiation in the apparatus of the invention having the internal spiral geometry. Each sector proceeding from left to right in FIG. 20 represents increased lamp wattage. For at least the first three sectors it can be seen that the fluid particle irradiation is controlled and equalized to be very uniform. Even the last sector, based on the highest lamp wattage, shows a generally uniform irradiation profile. Fluid particle irradiation being controlled and equalized advantageously results in efficient fluid particle irradiation and predictable increased fluid flow rates through the apparatus.

The invention is illustrated further by the following study of the bactericidal effect utilizing the invention as a nonthermal pasteurizer (SIAD TGIR) on bacterial contaminants in liquids. TGIR stands for Transportable Ground IRadiation. The purpose of this study was examine the effect of exposure time of the SIAD TGIR nonthermal pasteurizer on viable cell counts of previously reported bacterial contaminants in liquids.

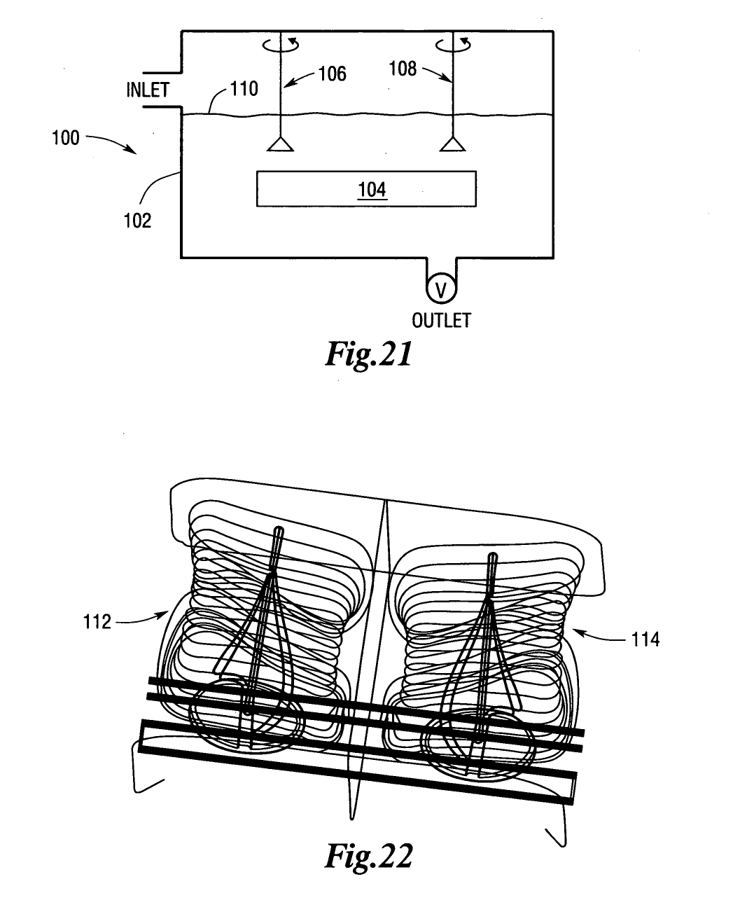

A non thermal pasteurizer according to the invention is illustrated in the schematic diagram of FIG. 21. Briefly, pasteurizer 100 includes a tank 102 into which a lamp 104 according to the invention is located. At least one motor driven mixing device is provided, in the present illustration two mixing devices 106, 108 are shown. Each device comprises a rod or shaft coupled at one end to a motor and provided at the end within tank 102 with a blade-like formation to provide a mixing action when the shaft is rotated. Liquid to be pasteurized is introduced to tank 102 as represented by the level designated 110 and remains therein for the predetermined pasteurization time. Lamp 104 is similar to lamp 10 shown in FIGS. 1-4 but does not have the threads 23. In this embodiment, which may be viewed as batch processing, the liquid flows around the exterior of lamp 104, the flow being a swirling type of flow promoted by the action of mixers 106, 1068. The mixers 106, 108 cause the fluid to go around and around lamp 104, and after a set time the fluid is drained from pasteurizer 100. Lamp 104 can be operated in the multi-band mode previously described, i.e. UV-A, UV-B and UV-C and in the output ratios about 35% UV-A, about 40% UV-B and about 25% UV-C.

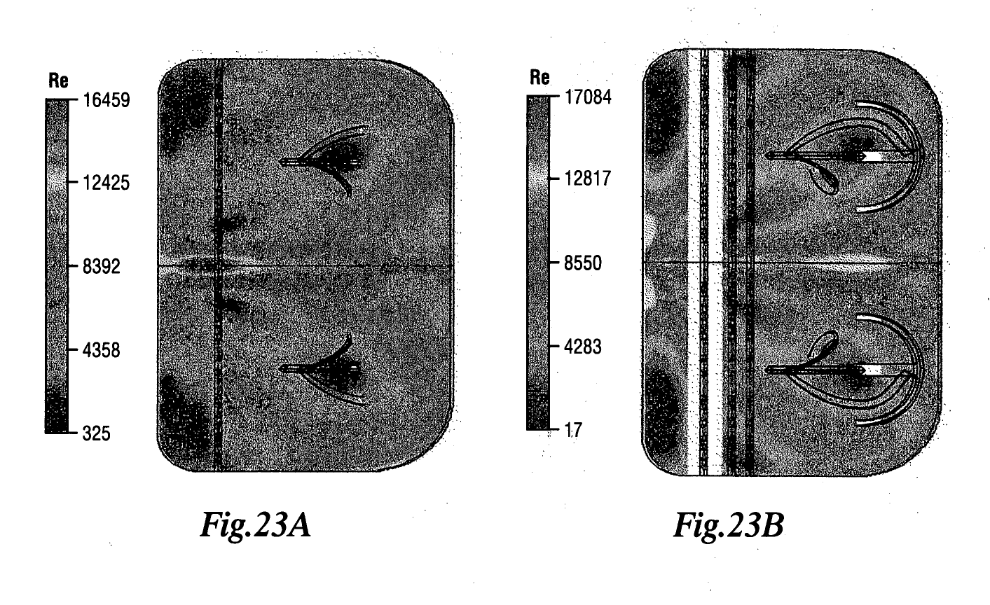

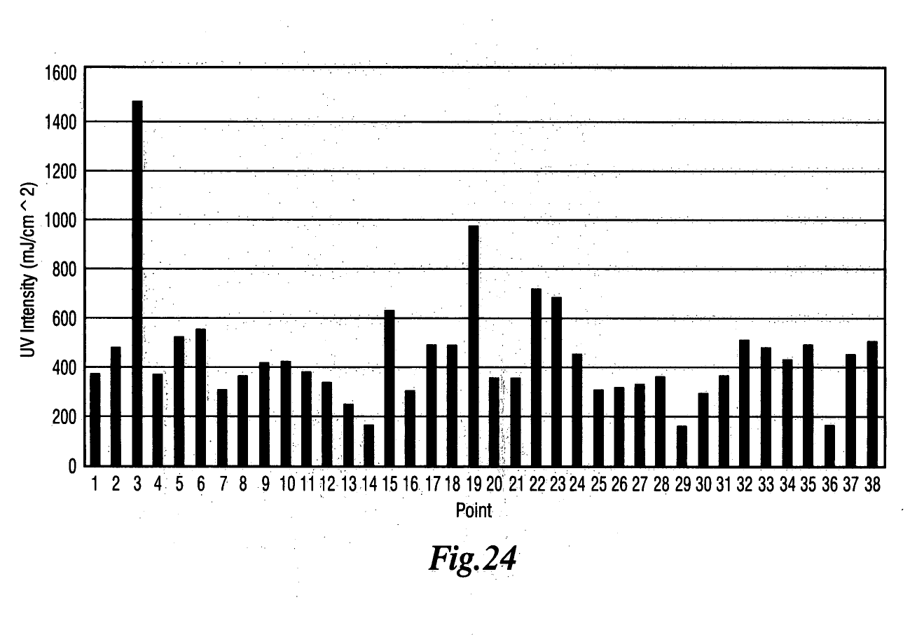

FIG. 22 is a schematic diagram illustrating particle path fluid dynamics in pasteurizer 100. Lines 112 and 114 represent the fluid particle flow paths associated with the two mixers. FIGS. 23A and 24B are graphs providing Reynolds Number plots associated with the illustration of FIG. 22. FIG. 24 is a graph showing particle irradiation in the pasteurizer illustrated in FIGS. 21 and 22 in the batch processing mode.

Included in the study were Salmonella choleraesuis subsp. choleraesuis serotype enteritidis ATCC strain 13076, Escherichia coli ATCC strain #43895 (O157:H7) and ATCC strain #47056, all acid tolerant, gram-negative, non-spore forming enteric rods. E. coli strain #47056 was used in some cases as a surrogate for ATCC #43895 the more pathogenic strain of E. coli. In addition, Bacillus cereus (ATCC #11778), a faculative, gram positive, spore forming rod, often used as a surrogate for Bacillus anthracis, was included.

Test strains maintained on the surface of Tryptic Soy Agar (TSA) were used to inoculate two to three liters of ½-strength Brain Heart Infusion broth. After 24 hours of incubation at 37° C. in air, the cells were harvested ascetically by centrifugation (15000 rpm for 20 min.), resuspended in sterile ¼ strength Ringer's solution, and used to inoculate seven liters of water, cider, and orange juice. After the SIAD TGIR nonthermal pasteurizer lamp reached a voltage of approximately 525 volts (˜1 minute) aliquots were ascetically removed at predetermined times as indicated in Tables 4-9, diluted to concentrations of 10⁻², 10⁻⁴ and 10⁻⁴, then plated onto TSA using a Spiral Plater (Spiral Systems Inc.) and incubated at 37° C. in air. Maximum voltage and liquid temperature were recorded at each sample time and the speed of the mixer was maintained at 530 rpm. After 24 hours, recoverable cell count was determined and compared to that of an equivalent, unexposed cell suspension of each strain.

### Water

Ringer's tablets were added to sterile distilled water to minimize cell lysis of the test strains due to the increased osmotic from the water. Enough tablets were added to give a final concentration ¼^{th} that of physiological saline.

Test strains: Escherichia coli (ATCC #47056), Bacillus cereus (ATCC #11778).

### Orange Juice

Freshly squeezed as well as juice with heavy pulp content (Tropicana) were used in the study.

Test strains: Escherichia coli (ATCC #47056), Salmonella choleraesuis subsp. choleraesius serotype enteritidis (ATCC #13076).

### Apple Cider

Fresh squeezed.

Test strain: Escherichia coli [ATCC #43895 (O157:H7)]

The test results for water are set forth in Tables 4 and 5.

### Water

TABLE 4
Effect of SIAD TGIR Nonthermal Pasteurizer
with constant stirring on 24 hr
Culture of Escherichia coli (#47056)
in ¼ strength Ringer's solution

| Exposure | | Max. Temp In Degrees | | Reduction in |
|---|---|---|---|---|
| (min.) | Volts | Centigrade | Cfu's/ml | Log Units |
| 0 | N/A | 26.5 | 8.33E+08 | N/A |
| 2 | 530 | 30.2 | 2.03E+01 | 7.61 |
| 4 | 529 | 32.1 | 0.00E+00 | 8.92 |
| 6 | 529 | 34.9 | 0.00E+00 | 8.92 |
| 8 | 529 | 37.5 | 0.00E+00 | 8.92 |
| 10 | 529 | 39.9 | 0.00E+00 | 8.92 |
| 15 | 529 | 48.9 | 0.00E+00 | 8.92 |

TABLE 5
Effect of SIAD TGIR Nonthermal Pasteurizer
with constant stirring on 24 hr
Culture of Bacillus cereus (ATCC #11778)
in ¼ strength Ringer's solution

| Exposure | | Max. Temp In Degrees | | Reduction in |
|---|---|---|---|---|
| (min.) | Volts | Centigrade | ML/ml | Log Units |
| 0 | N/A | 23 | 8.33E+08 | N/A |
| 2 | 525 | 25 | 1.63E+08 | 0.71 |
| 4 | 525 | 29 | 0.00E+00 | 8.92 |
| 6 | 530 | 30 | 0.00E+00 | 8.92 |
| 8 | 531 | 33 | 0.00E+00 | 8.92 |
| 10 | 533 | 35 | 0.00E+00 | 8.92 |
| 30 | 534 | 51 | 0.00E+00 | 8.92 |

The test results for orange juice are set forth in Tables 6-8.

### Orange Juice

TABLE 6
Effect of SIAD TGIR Nonthermal Pasteurizer
with constant stirring on 24 hr
Culture of Escherichia coli (#47056)
in orange juice with heavy pulp

| Exposure | | Max. Temp In Degrees | | Reduction in |
|---|---|---|---|---|
| (min.) | Volts | Centigrade | ML/ml | Log Units |
| 0 | N/A | 7 | 4.39E+07 | N/A |
| 20 | 529 | 25 | 8.13E+03 | 3.73 |
| 30 | 529 | 33 | 1.22E+02 | 5.56 |
| 40 | 531 | 41 | 0.00E+00 | 7.64 |
| 50 | 529 | 48 | 0.00E+00 | 7.64 |
| 60 | 529 | 55 | 0.00E+00 | 7.64 |

TABLE 7
Effect of SIAD TGIR Nonthermal Pasteurizer
with constant stirring on 24 hr
Culture of Salmonella choleraesuis subsp.
choleraesuis serotype enteritidis (ATCC #13076)
in orange juice with heavy pulp

| Exposure | | Max. Temp In Degrees | | Reduction in |
|---|---|---|---|---|
| (min.) | Volts | Centigrade | ML/ml | Log Units |
| 0 | N/A | 8 | 1.20E+07 | N/A |
| 10 | 527 | 19 | 2.03E+05 | 1.77 |
| 20 | 529 | 28 | 1.63E+04 | 2.87 |
| 30 | 530 | 35 | 1.34E+03 | 3.95 |
| 40 | 530 | 43 | 2.64E+02 | 4.66 |
| 50 | 530 | 52 | 0.00E+00 | 7.08 |
| 60 | 528 | 60 | 0.00E+00 | 7.08 |

TABLE 8
Effect of SIAD TGIR Nonthermal Pasteurizer
with constant stirring and bubbling carbon
dioxide gas on 24 hr Culture of
Salmonella cholersesuis subsp. choleraesuis
serotype enteritidis (ATCC #13076)
fresh squeezed orange juice

| Exposure | | Max. Temp In Degrees | | Reduction in |
|---|---|---|---|---|
| (min.) | Volts | Centigrade | ML/ml | Log Units |
| 0 | N/A | 2 | 3.66E+06 | N/A |
| 5 | 529 | 10 | 1.02E+06 | 0.56 |
| 10 | 530 | 16 | 4.27E+04 | 1.93 |
| 15 | 529 | 20 | 1.22E+04 | 2.48 |
| 20 | 529 | 24 | 1.04E+03 | 3.55 |
| 25 | 527 | 29 | 2.44E+02 | 4.18 |
| 30 | 527 | 33 | 4.07E+01 | 4.95 |
| 40 | 525 | 43 | 0.00E+00 | 6.56 |
| 50 | 524 | 51 | 0.00E+00 | 6.56 |
| 60 | 525 | 60 | 0.00E+00 | 6.56 |

The test results for apple cider are set forth in Table 9.

### Apple Cider

TABLE 9
Effect of SIAD TGIR Nonthermal Pasteurizer
with constant stirring on 24 hr
Culture of Escherichia coli (#43895) (O157:H7)
in Apple Cider

| Exposure | | Max. Temp In Degrees | | Reduction in |
|---|---|---|---|---|
| (min.) | Volts | Centigrade | ML/ml | Log Units |
| 0 | N/A | 7 | 1.82E+08 | N/A |
| 15 | 545 | 22 | 2.03E+01 | 6.95 |
| 20 | 546 | 29 | 4.27E+02 | 5.63 |
| 30 | 547 | 37 | 1.83E+02 | 6.00 |
| 40 | 544 | 46 | 2.03E+01 | 6.95 |
| 50 | 545 | 55 | 0.00E+00 | 8.26 |
| 60 | 544 | 64 | 0.00E+00 | 8.26 |

Escherichia coli (Table 4) and Salmonella choleraesuis subsp. choleraesuis serotype enteritidis (Table 5) levels in water (¼ strength Ringer's solution) were reduced by more than 5 log units after two minutes of exposure to the SIAD TGIR Nonthermal Pasteurizer and viable cells could not be recovered after four minutes. Escherichia coli (Table 6) and Salmonella choleraesuis subsp. choleraesuis serotype enteritidis (Table 7) levels in orange juice with heavy pulp (¼ strength Ringer's solution) were reduced 5.56 and 3.95 Log units respectfully after thirty minutes of exposure to the SIAD TGIR Nonthermal Pasteurizer and viable cells could not be recovered after forty and fifty minutes, respectively. Carbon dioxide gas was bubble through fresh squeezed orange juice to preserve nutritional content and to improve the taste of the juice. The data in Table 8 demonstrates that the addition of CO2 to the process had little if any change in bactericidal effect.

The level of the pathogenic strain of Escherichia coli (ATCC #43895) in apple cider was reduced by more than 5 Log units after 15 minutes of exposure and was not recoverable after fifty minutes of exposure.

In conclusion the SIAD TGIR nonthermal pasteurizer process of the invention achieved a 5 Log reduction in viable cell count with Escherichia coli (ATCC #47056) in orange juice and water, with Escherichia coli [ATCC #43895 (O157:H7)] in apple cider, with Salmonella choleraesuis subsp. choleraesuis serotype enteritidis (ATCC #13076) in orange juice, and with Bacillus cereus (ATCC #11778) in water and was able to reduce the level of all test organisms to undetectable levels over time.

By way of example, in an illustrative pasteurizer of the invention, using a 1500 watt lamp of the invention, FDA 5 log reduction was achieved in about 25 minutes on 6 gallons of orange juice and in about 2 minutes on 6 gallons of water. No nutritional changes in either the orange juice or the water were observed. Using a 4 inch, 400 watt lamp of the invention, FDA 5 log reduction was achieved in about 20 seconds on 3 quarts of water. Again, no nutritional changes were observed.

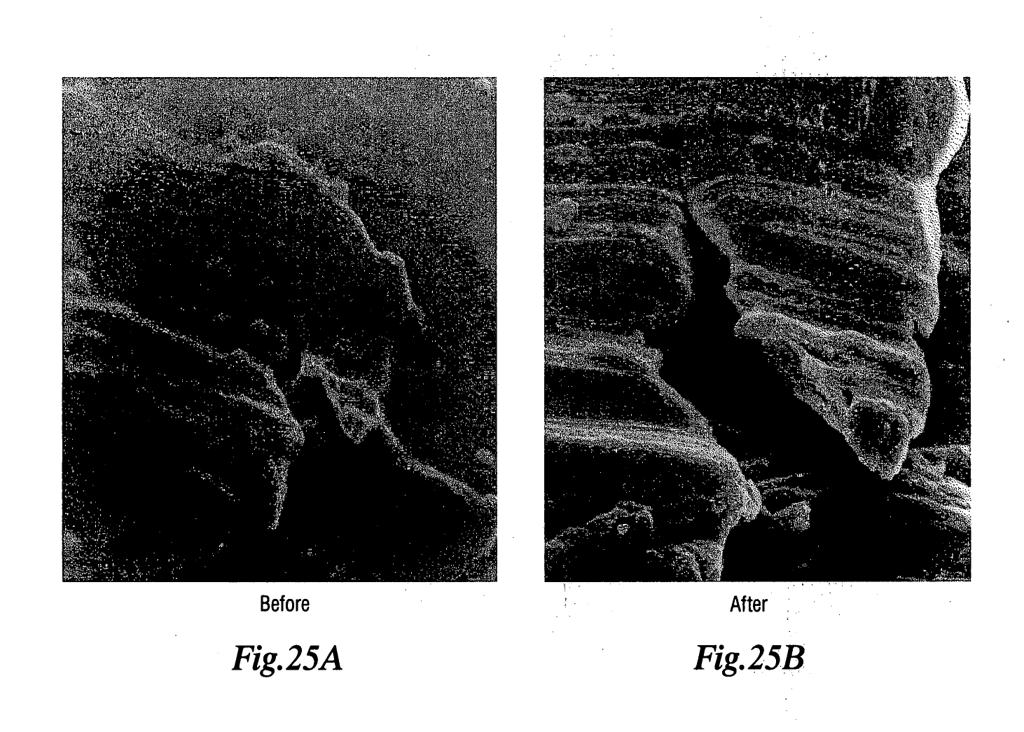

FIGS. 25A and 25B are scanning electron microscope photographs further illustrating the invention. In particular, they show the effect of a 10-second treatment of bacillus subtilis spores using the process of the invention. The spherical shaped spores are seen in FIG. 25A before application of the process of the invention. The process of the invention, utilizing UV-A, UV-B and UV-C for a duration of 10 seconds, completely destroyed the spores, reducing the microorganism to deposits of carbon, as evidenced by the absence of the spores in the photograph of FIG. 25B.

The invention is illustrated further by a nutrient analysis of orange juice and apple cider before and after treatment by the method of the invention. Many nutrients can be affected by irradiation and temperature. Both of these might be important components of the system and method of the invention. After the conditions needed for biological decontamination of the juices being studied are determined, samples that have been treated by the system and method of the invention will be analyzed for nutrient content. The nutrients that will be measured are those with potential to be damaged by the high intensity photonics and rapid heat transfer. In addition, only those nutrients that are present in significant levels in orange juice will be studied. The vitamins of interest in orange juice (per 8 fl oz, 116 kcal) for this project include Vitamin C (125 mg, 200% DRI), Thiamin (0.2 mg, 15% DRI), Riboflavin (0.074 mg, 5% DRI), and Folate (75 ug, 20% DRI).

Vitamin C (L-ascorbic acid and dehydro-L-ascorbic acid) is an essential nutrient, first related to the ancient disease scurvy. It is reported to be the basis of the first clinical trial on the lack of food component and disease by James Lind in 1753. It functions in the human as an antioxidant and as an enzyme cofactor for such reactions as collagen synthesis, carnitine and norepinephrine synthesis and liver detoxification. The current DRI for Vitamin C ranges from 15 mg/d for children aged 1 to 3 years to 120 mg/d for lactating women with an additional 35 mg/d for smokers. Degradation of L-ascorbic acid occurs in aqueous solutions in several conditions; low pH, high temperature, presence of oxygen and metals (copper as an example). Orange juice is an excellent media for preserving vitamin C if temperature is kept low and light is excluded, since the pH is low. Fresh orange juice is also an excellent source due to the lack of heat processing. The exposure to light in the process of the invention is an important issue. The first degradation reaction of L-ascorbic acid is to dihydroascorbic acid (DHAA). DHAA may then be irreversibly converted to 2,3 keto-L-gluonic acid.

Thiamin was one of the first of the water soluble vitamins to be identified, being an important cofactor for many enzymes (several involving energy metabolism) and also having some non-coenzyme functions. Diseases related to the deficiency of thiamin have been known for thousands of years, in particular beriberi. Oranges can be a significant source of thiamin, there being almost as much thiamin in a single medium orange as in a slice of enriched white bread. Thiamin is unstable in the presence of oxygen and heat; however its stability is better when pH is less than 7 as in orange juice. So a low temperature process in the absence of oxygen could be the best situation to preserve thiamin in aqueous solutions such as orange juice.

Riboflavin, as known as B₂, is an integral part of two key coenzymes (FAD and FMN). As FAD and FMN, riboflavin is crucial for energy metabolism, drug metabolism, and antioxidant functions. Riboflavin is stable to heat sterilization, but is very sensitive to light and oxidation. Light therapy for some newborns is known to cause riboflavin deficiency. The sensitivity to light has been a major reason for discontinuing the use of glass milk bottles with up to half the riboflavin being lost with 2 hours of exposure to bright sunlight. This sensitivity of light is related to production of reactive free radicals which react with riboflavin. As with thiamin, the level in a single orange is not much different than a slice of enriched white bread.

Folic acid or folate occurs in many different forms making it difficult to measure accurately, such as 5-methyl-FH₄ and 10 formyl-FH₄. In addition, folate also has various degrees of polyglutamine. Orange juice has a mix of folate glutamate, mono, and penta being the most common. This makes it imperative to measure folate by a bioassay. Folate is critical for nucleic acid and protein metabolism with several well-known deficiency diseases, such as megaloblastic anemia. Folate, of all of the other vitamins studied, is very liable to UV light, oxygen, and heat. It is most sensitive to light in the presence of oxygen. Oxidation leads to the conversion of FH₄ to dihydrofolic acid and fully oxidized folate. Eventually the process of oxidation leads to inactive forms. This is especially true in acidic media where the inactive 5-methyl-5,8-FH₂. Therefore it may be essential to avoid the presence of oxygen to preserve the active folic in foods.

Fresh orange juice can be an important source of three of these vitamins: C, Thiamin, and Folate. The fourth vitamin, riboflavin, even though not present at significant dietary levels, was studied due to its sensitivity to oxidation. The results could be useful in determining the effect of the system and method of the invention on other nutrients and oxidation in general. Therefore it was decided that it was necessary that these vitamins be assayed before and after treatment using the system and method of the invention.

A vitamin analysis was first carried out on apple juice and orange juice without CO₂ being present. Folate, thiamin, riboflavin, and vitamin C were analyzed in both Orange and Apple Juice. Vitamin C was measured by a fluorometric assay that measures both the oxidized and reduced forms of Ascorbic acid. Reference may be made to Deutsch, M. J. (Assoc. Ch. ed., 1990) “Vitamins and other Nutrients,” Chapter 45 in Hlerich, K. (ed.) Official Methods of Analysis of the Association of Official Analytical Chemists. 15^{th} Edition Volume 2. Association of the Official Analytical Chemists, Inc. Arlington, Va., Method No. 967.22 “Vitamin C (Ascorbic Acid) in Vitamin Preparations: Microfluorometric Method,” 1059-1060. [AOAC 967.22]

In brief, samples are homogenized then extracted with 4% (w/v) Metaphosphoric acid—MeOH pH 2.1. After being filtered through activated charcoal, a subsample is treated with boric acid-sodium acetate to prepare a blank while another portion is treated with sodium acetate. These samples are reacted with o-phenylenediamine to produce a fluorophor. After standing for 35 min. (protected from light) fluorescence is measured. Thiamine was measured by the fluorometric assay [AOAC 942.23]. Reference may be made to Deutsch, M. J. (Assoc. Ch. ed., 1990). “Vitamins and Other Nutrients,” Chapter 45 in Hehich, K. (ed) Official Methods of Analysis of the Association of Official Analytical Chemists, 15^{th} Edition Volume 2. Association of Official Analytical Chemists, Inc. Arlington, Va., Method No. 942.23 “Thiamine in Foods: Fluorometric Method.” Samples are homogenated before analysis. In brief, NaCl or KCl was mixed with the standard or sample solution into a reaction vessel. Alkaline KFe (CN) is then added and gently swirling by a rotary motion. Then isobutanol is added to the reaction vessel, which is stoppered and shaken vigorously followed by centrifugation at low speed. The lower phase is removed and anhydrous sodium sulfate is added to the isobutanol layer with vortexing and shaking. The fluorescence of isobutanol extract is measured using a fluorometer. Riboflavin content was determined by a microbiological assay. Reference may be made to Baker, H., and Frank O. in Riboflavin, Rivlin, R. S. (ed) Plenum Press, NY: p 49.

Samples are autoclaved with 0.1 HCl to liberate the flavins. Samples are then filtered. The growth of Lactobacillus casei is used as the endpoint. Folate was analyzed by a microbiological assay using Lactobacillus casei. Reference may be made to Wight, A. J. A. and Phillips, D. R. Brit. J. Nutr., 53:569 (1985).

Juice samples were Tropicana Pure Premium Orange Juice (not from concentrate) with lots of pulp and Tops brand Walter 100% Apple Juice. The juice samples were refrigerated (5 C) before treatment by the system and method of the invention. Samples were collected from the treatment at three time points: first after mixing, second after 20 minutes and finally after 40 minutes. After collection samples were chilled to 5 C before being frozen at −20 C.

Assayed folate increased during the process in Apple juice and during the first 20 minutes in the Orange Juice but decreased to below detectable limits in the final sample at 40 minutes. These results could be due to an increased solubility of this nutrient. The eventual loss of folate after 40 minutes may be the result of either the presence of oxygen or the increased temperature or both. Samples increased in temperature during the process from around 12 C to 50 C by 40 minutes.

Apple juice had no detectable riboflavin or vitamin C. There was a significant loss of Vitamin C and Riboflavin in the Orange Juice during the process. This is probably due to the oxygen and heat. The results are summarized in Table 10.

TABLE 10
Apple Juice

| | | 20 minutes | 40 minutes |
|---|---|---|---|
| Per 100 g | Before 11 C. | 27 C. | 45 C. |
| Folate, ug | 3.83 | 10.5 | 22.7 |
| Thiamine, ug | 10 | 30 | 10 |
| Riboflavin, ug | <20* | <20* | <20* |
| Vitamin C, mg | <1* | <1* | <1* |

Orange Juice

| | | 20 minutes | 40 minutes |
|---|---|---|---|
| Per 100 g | Before 13 C. | 30 C. | 49 C. |
| Folate, ug | 26.3 | 47.4 | <6* |
| Thiamine, ug | 60 | 60 | 40 |
| Riboflavin, ug | 30 | 20 | <20* |
| Vitamin C, mg | 27.9 | 12.0 | 1.1 |

*Values below detectable limits.

Next, a vitamin analysis was carried out on orange juice with co₂ being present. Juice samples were Jennings Citrus fresh squeezed—not pasteurized—orange juice. The juice samples were refrigerated (5 C) before treatment by the system and method of the invention. Samples of Jennings Citrus orange juice were collected at 0, 25 and 45 minutes during treatment. Samples were refrigerated (5 C) then frozen (−20 C) before analysis. All the vitamins were analyzed by the same methodology except vitamin C which analyzed by a different procedure than done with the non-CO2 samples. Total ascorbic acid was determined by the dinitrophenylhydrazine method. Reference may be made to Burtis C. and Ashwood E. (eds); Dinitrophenyl hydrazine Method; Tietz Textbook of Clinical Chemistry 2^{nd} Edition; W.B. Saunders Co.; 1994; pp 1313-1314. 2,4-dinitrophenylhydrazine, ascorbic acid, thiourea and CuSO₄ were purchased from Sigma. Sulfuric and meta-phosphoric acid (MPA) were purchased from J. T. Baker (Phillipsburg, N.J.). Samples for analysis were stabilized by adding 0.5 mL juice sample to 2.0 ml 6% MPA and centrifuging at 3,000×g for 10, minutes. Clear supernatant was aspirated for batch analysis. Standards encompassing the range from 0-2.0 mg/dL ascorbic were prepared fresh for each analysis in 6% MPA. Color reagent was prepared prior to each analysis by mixing 100 mL dinitrophenylhydrazine (2.0 g/dL in 4.5 M sulfuric acid), 5.0 mL CuSO₄ (0.6 g/dL in H₂O) and 5.0 mL thiourea (5.0 g/dL in H₂O). For assay, MPA supernatants were diluted by 10-fold serial dilution and 0.6 mL of each dilution (1:1 to 1:10,000) or standard in MPA was mixed with 0.2 mL color reagent and incubated at 37° C. for 3 hours. Samples were chilled on ice for 10 minutes and then vortexed during addition of 1.0 mL 12M H₂SO₄. Care was taken to insure that the temperature of the sample did not exceed room temperature. Absorbance of each sample was determined at 520 nm on a shimadzu 160 UV spectrophotometer and plotted against concentration. The results are summarized in Table 11.

TABLE 11
Orange Juice with CO₂

| | Before | 20 minutes | 40 minutes |
|---|---|---|---|
| Per 100 mL | 13 C. | 31 C. | 45 C. |
| Folate, ug | 28.2 | 32.4 | 24.3 |
| Thiamine, ug | 70 | 80 | 70 |
| Riboflavin, ug | 30 | 30 | 20 |
| Vitamin C, mg | 48 | 48 | 50 |

None of the four vitamins that were measured showed a large drop in their content with the treatment by the system and method of the invention with CO₂.

From the foregoing it is apparent that the invention accomplishes its intended objectives. While embodiments of the invention have been described in detail, that has been done for the purpose of illustration, not limitation.

1. Apparatus for disinfection/pasteurization of fluids having micro-organisms, the apparatus comprising:
a) a photonic lamp enclosed in a quartz envelope for emitting ultraviolet light;
b) an ozone free, metallic doped quartz enclosure for the lamp; and
c) a vessel containing the lamp in an enclosure and a first in-line stationary spiral lead surrounding the enclosure and the vessel having an inlet, an outlet and a chamber in fluid communication therewith defining a flow path for fluid to be disinfected/pasteurized; and
d) at least one turbulence bar joined to the first spiral lead and the turbulence bar for creating turbulent flow in the vessel to mix the fluid so that the micro-organisms are exposed to the ultraviolet light when the lamp is in the on position and reduced.

2. Apparatus according to claim 1, wherein the lamp is in the form of a tube and the enclosure and the vessel are generally cylindrical in shape, with the lamp, enclosure, and vessel being in generally concentric relation.

3. Apparatus according to claim 2, wherein the inlet and outlet are at opposite ends of the vessel.

4. Apparatus according to claim 2, wherein the diameter of the vessel is about twice the diameter of the enclosure, and wherein the diameter of the enclosure is about twice the diameter of the lamp.

5. Apparatus according to claim 1, wherein the photonic lamp provides ultraviolet radiation having wavelength bands designated UV-A, UV-B and UV-C wherein band UV-A has a wavelength from about 315 nanometers to about 400 nanometers, band UV-B has a wavelength from about 280 nanometers to about 315 nanometers and band UV-C has a wavelength from about 100 nanometers to about 280 nanometers.

6. The apparatus according to claim 1 wherein the lamp emits UV light at a wavelength in a range of 2200 A to 2250 A in order to turn the riboflavin in the micro-organisms into free radicals to render the micro-organism susceptible to self destruction or destruction by an external force.

7. The apparatus according to claim 1 wherein the lamp emits UV light at 3710 A in order to turn the riboflavin in the micro-organisms into free radicals rendering to render the micro-organism susceptible to self destruction or destruction by an external force.

8. The apparatus according to claim 1 wherein the lamp emits UV light at 4440 A in order to turn the riboflavin in the micro-organisms into free radicals to render the micro-organism susceptible to self destruction or destruction by an external force

9. The apparatus according to claim 1 wherein the lamp emits UV light at 4750 in order to turn the riboflavin in the micro-organisms into free radicals to render the micro-organism susceptible to self destruction or destruction by an external force.

10. Apparatus according to claim 1, wherein the lamp operates at a temperature ranging from about 600 degrees centigrade to about 800 degrees centigrade.

11. Apparatus according to claim 1, wherein the enclosure an ozone free, metallic doped quartz and the enclosure allows transmission of ultraviolet radiation from the lamp into the fluid.

12. Apparatus according to claim 1, wherein further wherein the at least one lead includes multiple in-line spirals leads.

13. Apparatus according to claim 1 wherein the at least one lead abuts against the vessel and abuts against the enclosure, and a flow path is defined by the at least one lead, the vessel and the enclosure

14. The apparatus according to claim 13 wherein the at least one turbulence bar extends into the flow path and is joined to an outer edge of the at least one lead and the turbulence bar is for causing turbulent flow in the vessel such that the fluid is mixed such that the micro-organisms are exposed to the ultra violet light.

15. A method for disinfection/pasteurization of fluids, the method comprising:
a) providing a photonic lamp enclosed within an ozone free metallic doped quartz envelope;
b) providing an ozone free, metallic doped quartz enclosure for the lamp;
c) providing a vessel containing the lamp, enclosure and an in-line stationary spiral lead surrounding the enclosure, the vessel having an inlet, an outlet and a chamber in fluid communication therewith defining a flow path for fluid to be disinfected/pasteurized;
d) operating the lamp to introduce ultraviolet radiation and heat from the lamp into the fluid; and,
e) providing at least one turbulence bar and joining the turbulence bare to the first spiral lead and the turbulence bar for creating turbulent flow in the vessel to mix the fluid so that the micro-organisms are reduced.

16. The method according to claim 15 further including using the lamp to emit UV-A in a range of 2200 A to 2250 A in order to turn the riboflavin in the micro-organisms into free radicals rendering the micro-organism susceptible to self destruction or destruction by an external force.

17. The method according to claim 15 wherein further including using the lamp to emit UV light at 2660 A in order to turn the riboflavin in the micro-organisms into free radicals rendering the micro-organism susceptible to self destruction or destruction by an external force.

18. The method according to claim 15 further including using the lamp to emit UV light at 3710 A in order to turn the riboflavin in the micro-organisms into free radicals rendering the micro-organism susceptible to self destruction or destruction by an external force.

19. The method according to claim 15 further including using the lamp to emit UV light at 4440 A in order to turn the riboflavin in the micro-organisms into free radicals rendering the micro-organism susceptible to self destruction or destruction by an external force.

20. The method according to claim 15 further including using the lamp to emit UV light at 4750 A in order to turn the riboflavin in the micro-organisms into free radicals rendering the micro-organism susceptible to self destruction or destruction by an external force.

21. The method according to claim 15, further including operating the lamp at a temperature ranging from about 600 degrees centigrade to about 800 degrees centigrade.

22. The method according to claim 15, wherein the enclosure an ozone free, metallic doped quartz and the enclosure allows transmission of ultraviolet radiation from the lamp into the fluid.

23. The method according to claim 15, further including providing the at least one lead to have multiple in-line spirals leads.

24. The method according to claim 15 wherein the at least one lead abuts against the vessel and abuts against the enclosure, and defining a flow path between the at least one lead, the vessel and the enclosure

25. The method according to claim 24 further including extending the at least one turbulence bar into the flow path and joining the turbulence bar to an outer edge of the at least one lead and the turbulence bar is for causing turbulent flow in the vessel such that the fluid is mixed such that the micro-organisms are exposed to the ultra violet light.

26. A method for disinfection/pasteurization of fluids having micro-organisms, the method comprising:
a) providing a fluid for disinfection/pasteurization;
b) providing a photonic lamp and exposing the fluid to ultraviolet light having at least one of the following wavelengths: 2200 A-2250 A, 2660 A, 3710 A, 4440 A, and 4750 A;
c) allowing the riboflavin in the micro-organisms to absorb the ultraviolet light and form free radicals; and
d) allowing the free radicals formed from process of disassembling of riboflavin to disrupt cellular metabolic activity and structure of the micro-organisms.

27. The method according to claim 26 further including allowing the free radicals to interfere with the quality control function of protein in the cells of the micro-organisms to render the cell susceptible to either self destruction or destruction by an external force.
